(19) United States
(12) Patent Application Publication
(10) Pub. No.: US 2026/0364343 A1
(43) Pub. Date: Nov. 19, 2026

(54) MUSCLE STIMULATION DEVICE

(71) Applicant: CHIRP INNOVATION, LLC., Lehi, UT (US)

(72) Inventors: Tate Sherman Stock, Draper, UT (US); Weston N. Hills, Kamas, UT (US); Brian Connolly Nielson, American Fork, UT (US); Reeve Stewart Sudweeks, Lehi, UT (US); Caden-Thomas Atwood Wakefield, Edgewood, WA (US); Jason Lee Walton, Draper, UT (US)

(21) Appl. No.: 19/210,720

(22) Filed: May 16, 2025

(51) Int. Cl. A61N 1/08; A61N 1/36; G08B 5/36

(52) U.S. Cl. CPC A61N 1/08; A61N 1/36003; A61N 1/36021; G08B 5/36

(57) ABSTRACT

A system includes a remote control and a stimulation device with a pulse generator. The stimulation device is configured to receive data from the remote control associated with providing electrical stimulation, determine, based at least in part on the data, a setting associated with providing the electrical stimulation, and generate, based at least in part on the setting and via the pulse generator, electrical pulses to provide the electrical stimulation.

### BACKGROUND

Devices and apparatuses exist to treat pain and discomfort, such as myofascial pain. In some examples, devices may employ transcutaneous electrical nerve stimulation (TENS) or electrical muscle stimulation (EMS) to relieve pain and/or stimulate muscles. However, conventional devices that utilize TENS and/or EMS are often large in size, difficult to transport, tedious to operate, and/or ineffective.

### BRIEF DESCRIPTION OF THE DRAWINGS

The detailed description is described with reference to the accompanying figures. In the figures, the left-most digit(s) of a reference number identifies the figure in which the reference number first appears. The use of the same reference numbers in different figures indicates similar or identical components or features. The systems depicted in the accompanying figures are not to scale and components within the figures may be depicted not to scale with each other.

FIG. 1 illustrates an example system that provides transcutaneous electrical nerve stimulation (TENS) and/or electrical muscle stimulation (EMS), according to an embodiment of the present disclosure.

FIG. 2 illustrates select components of the system of FIG. 1, according to an embodiment of the present disclosure.

FIG. 3 illustrates a front view of an example remote control of the system of FIG. 1, according to an embodiment of the present disclosure.

FIG. 4 illustrates a back view of the remote control of FIG. 3, according to an embodiment of the present disclosure.

FIG. 5A illustrates a front view of an example stimulation device of the system of FIG. 1, according to an embodiment of the present disclosure.

FIG. 5B illustrates a back view of the stimulation device of FIG. 5A, according to an embodiment of the present disclosure.

FIG. 6 illustrates a side view of the stimulation device of FIG. 5A, according to an embodiment of the present disclosure.

FIG. 7 illustrates a front view of an example case of the system of FIG. 1, according to an embodiment of the present disclosure.

FIG. 8 illustrates a side view of the case of FIG. 7, according to an embodiment of the present disclosure.

FIG. 9A illustrates an example receptacle of the case of FIG. 7 to receive the stimulation device of FIG. 5A, according to an embodiment of the present disclosure.

FIG. 9B illustrates the receptacle of FIG. 9A, according to an embodiment of the present disclosure.

FIG. 10 illustrates an example receptacle of the case of FIG. 7 to receive the remote control of FIG. 3, according to an embodiment of the present disclosure.

FIG. 11 illustrates a top view of an example pad that may be used in conjunction with the stimulation device of FIG. 5A, according to an embodiment of the present disclosure.

FIG. 12 illustrates a cross-sectional view of the pad of FIG. 11, taken along line A—A of FIG. 11, according to an embodiment of the present disclosure.

FIG. 13 illustrates example placements of the pad of FIG. 11 on a user, according to an embodiment of the present disclosure.

FIG. 14 illustrates an example pad that may be used in conjunction with the stimulation device of FIG. 5A, according to an embodiment of the present disclosure.

FIG. 15 illustrates an example cable that connects the stimulation device of FIG. 5A to the pad of FIG. 14, according to an embodiment of the present disclosure.

FIG. 16 illustrates an example placement of the pad of FIG. 14 on a user, and a connection between the pad of FIG. 14 with the stimulation device of FIG. 5A via the cable of FIG. 15, according to an embodiment of the present disclosure.

FIG. 17 illustrates an example pad that may be used in conjunction with the stimulation device of FIG. 5A, according to an embodiment of the present disclosure.

FIG. 18 illustrates an example placement of the pad of FIG. 17 on a user, according to an embodiment of the present disclosure.

FIG. 19 illustrates an example pad that may be used in conjunction with the stimulation device of FIG. 5A, according to an embodiment of the present disclosure.

FIG. 20 illustrates an example placement of the pad of FIG. 19 on a user, according to an embodiment of the present disclosure.

FIG. 21 illustrates an example pad that may be used in conjunction with the stimulation device of FIG. 5A, according to an embodiment of the present disclosure.

FIG. 22 illustrates an example placement of the pad of FIG. 21 on a user, according to an embodiment of the present disclosure.

FIG. 23 illustrates an example pad that may be used in conjunction with the stimulation device of FIG. 5A, according to an embodiment of the present disclosure.

FIG. 24 illustrates an example placement of the pad of FIG. 23 on a user, according to an embodiment of the present disclosure.

FIG. 25 illustrates an example pad that may be used in conjunction with the stimulation device of FIG. 5A, according to an embodiment of the present disclosure.

FIG. 26 illustrates an example placement of the pad of FIG. 25 on a user, according to an embodiment of the present disclosure.

FIG. 27 illustrates an example pad that may be used in conjunction with the stimulation device of FIG. 5A, according to an embodiment of the present disclosure.

FIG. 28 illustrates an example placement of the pad of FIG. 27 on a user, according to an embodiment of the present disclosure.

FIGS. 29-31 illustrate example user interfaces (UIs) associated with providing TENS and/or EMS using the system of FIG. 1, according to an embodiment of the present disclosure.

FIG. 32 illustrates an example process for providing TENS and/or EMS, according to an embodiment of the present disclosure.

FIG. 33 illustrates an example process for providing TENS and/or EMS, according to an embodiment of the present disclosure.

### DETAILED DESCRIPTION

This application is directed, at least in part, to a stimulation device configured to provide therapeutic relief to a user, according to an embodiment of the present disclosure. The stimulation device may deliver electrical pulses to the user for transcutaneous electrical nerve stimulation (TENS) or electrical muscle stimulation (EMS). In some instances, a remote control may be used to control the stimulation device to adjust the intensity, duration, and/or frequency of the electrical pulses. Moreover, the remote control may be used to control more than one of the stimulation devices, whether independently or in coordination with one another. The stimulation devices may be applied to the user via pads. For example, the pads may include an adhesive material for adhering to the user. In addition, the pads may include electrodes for delivering the electrical pulses to the user and connectors for coupling to, or receiving, the stimulation device. The stimulation devices, the remote control, and the pads may be compactly stored in a case. In some instances, the stimulation devices, the remote control, the pads, and the case may be referred to as an assembly or a system.

The stimulation device may include a pulse generator that generates the electrical pulses. The pulse generator is configured to generate the electrical pulses (e.g., current) according to any desired duration, interval, timing, frequency, amplitude, intensity, etc. The pulse generator, or the electrical pulses generated by the pulse generator, may be controlled, for example, via the remote control. The stimulation device may include connectors that connect with corresponding connectors on the pads to deliver the electrical pulses to the user. The stimulation device may also include connector pads that connect with connector pins of the case. A battery of the stimulation device may be charged via an engagement between the connector pads and the connector pins (i.e., when stored in the case).

The stimulation device may have any shape. For example, in some instances, the stimulation device may be circular in shape. However, other shapes (e.g., square, hexagon, etc.) are envisioned. In some instances, the stimulation device includes input/output components, such as lighting elements, buttons, etc., that may be used to control an operation of the stimulation device, indicate an operational status of the stimulation device, and so forth. For example, the lighting elements may be arranged as a light ring and may output light associated with the electrical pulses (e.g., duration, intensity, etc.), an operational status of the stimulation device (e.g., on/off, awaiting to pair, paired, etc.). The button, for example, may be used to power on/off the stimulation device, pair the stimulation device with the remote control, and so forth. In some instances, the light ring may be disposed around the button. The stimulation device may also include ports or other connectors, such as a USB-C connector, for charging the stimulation device and/or connecting the stimulation device to other stimulation devices and/or pads (e.g., via wires, cables, etc.).

The remote control, as introduced above, may be used to control one or more operations of the stimulation devices. In some instances, the remote control may include one or more input/output components, such as a display, buttons (e.g., D-pad, toggle, etc.), lighting elements, and so forth. The buttons of the remote control may be used to select various modes, operations, etc., of the stimulation device, or the electrical pulses delivered by the stimulation device. For example, the stimulation devices may be controlled to various modes, where the modes may be associated with different durations of the electrical pulses, intensity of the electrical pulses, frequency of the electrical pulses, patterns of the electrical pulses, and so forth. Generally, the modes may be associated with setting(s), where the setting(s) may be used to control the stimulation device. For example, depending upon the desired or selected mode, the stimulation device may be controlled according to the setting(s) and/or the electrical pulses may be generated in accordance with the setting(s). The display may also display information such as whether the remote control is paired to the stimulation devices, a battery level (e.g., charge) of the remote control and/or the stimulation devices, a mode or a setting of the stimulation device, a duration of the electrical pulses, a time, and so forth.

The display may output information associated with the modes, the electrical pulses, the setting(s), etc. For example, the display may display information of the selected mode (e.g., massage, pain relief, stretch, etc.), the intensity of the electrical pulses, the duration (or remaining duration) of the electrical pulses, etc. The user may utilize the buttons, for example, to toggle between the modes, adjust the setting(s), etc., which may be used to update the display. When a desired mode and/or setting(s) are selected, corresponding data, signals, etc., may be wirelessly transmitted to the stimulation device(s), whereby the stimulation device(s) may output the electrical pulses in accordance with the mode and/or setting(s). The user may continuously switch between mode(s) and/or adjust the setting(s).

The remote control may communicatively couple to any number of the stimulation devices, such as one, two, three, etc. In some instances, the stimulation devices may be controlled independently or dependently of one another. For example, each stimulation device may be independently controlled to operate at a certain mode and/or settings. In doing so, the stimulation devices may provide different TENS and/or EMS to different portions of the user. Alternatively, the stimulation devices may operate in coordination with one another. For example, the electrical pulses generated across the stimulation devices may be coordinated, synchronized, timed with one another, output in parallel, series, etc.

The remote control may include a battery that is charged via an engagement between connector pads of the remote control and connector pins of the case (i.e., when stored in the case). Network interface(s) of the remote control may be used to wirelessly communicate with corresponding network interface(s) of the stimulation devices. The remote control and the stimulation devices may wirelessly communicate over any suitable network(s), such as Bluetooth, Wi-Fi, Cellular, Zigbee, etc. The stimulation device may include corresponding network interface(s) to communicate with the remote control.

The pads may include connectors that connect with the connectors of the stimulation devices. In some instances, the connectors may include magnetic and/or ferrous materials that allow the stimulation devices to secure to the pads. An engagement between the connectors of the pad and the connectors of the stimulation device permits the electrical pulses generated by the stimulation device to be administered (e.g., transferred) to the user. For example, the connectors of the pad may connect to electrodes that are in contact with or disposed adjacent to the skin of the user (e.g., via wires). The skin of the user completes a circuit between the electrodes, thereby allowing the electrical pulses (i.e., current) to be administered to the user. More particularly, as the pulse generator of the stimulation device generates electrical pulses, current may flow from a first of the connectors of the stimulation device, to a first of the connectors of the pad, to a first of the electrodes on the pad, into/through the user, to a second of the electrodes on the pad, to a second of the connectors of the pad, and to a second of the connectors of the stimulation device. Completion of this circuit allows TENS and/or EMS to be delivered to the user.

The pads may be manufactured from one or more layers. For example, a first layer may be manufactured from a first material (e.g., neoprene, rubber, etc.) and a second layer may be manufactured from a second material (e.g., conductive hydrogel, hydrocolloid, urethane, etc.) different than the first material. The connectors may be disposed adjacent to the first material of the pads, while the electrodes may be disposed adjacent to, or within, the second material of the pads. The first material and the second material may be flexible to conform to portions of the user. The second material may allow for the pad to be adhered to the user. In addition, the second material may be conductive to deliver the electrical pulses to the user. The electrodes of the pad may be separated via a void, channel, etc., in the second material.

The pad may be removed from the user after use. The pads, from time to time, may be replaced as the adhesiveness of the second material is reduced. Alternatively, adhesives may be reapplied to the second layer of the pads.

The pads may include any suitable shape. In some instances, the shape of the pad may be based on the location at which the pads are adhered to the user. For example, pads may be designed for the thighs, back, arms, neck, calves, shoulders, etc., of the user. In such instances, the pads may vary in shape, design, etc. As an example, the pads may have different arms, appendages, cut-outs, reliefs, etc., to permit the pads to be applied to the user. In addition, these different arms, appendages, cut-outs, reliefs, etc., permit the pads to conform to the user. The pads may also be of different sizes. Regardless, the pads include the connectors and the electrodes for administering the electrical pulses.

In some instances, the pads may be configured to receive more than one of the stimulation devices. In such instances, the pads may have more than one set of electrodes. For example, a pad may include two sets of connectors, one for connecting to a first stimulation device and a second for connecting to a second stimulation device. The two sets of connectors may respectively connect to electrodes. Moreover, noted above, the first stimulation device and the second stimulation device may be controlled independently or dependently, whether on the same pad or different pads. Additionally, the electrodes may be separate from one another to permit the electrical pulses to be delivered to the user.

The pads may include an indication associated with their placement and/or function. For example, the pads may include an indication whether the pad is designed for or to be utilized on the back, the neck, the shoulder, etc. The pads may also include tabs that permit the pads to be easily removed from the user. The tabs may not include the second material (e.g., adhesive) to permit the user to grasp and remove the pad.

The case is configured to store the stimulation devices, the remote control, the pads, etc., when not in use, for charging, etc. In some instances, the case includes receptacles for the stimulation devices, a receptacle for the remote control, and/or a compartment for the pads. The compartment may be used to store the pads. In some instances, the compartment may include, or be formed from, a webbing or other material behind which the pads are stored.

The case includes the connector pins, which may be disposed within the receptacles to charge the stimulation devices and the remote control, respectively. For example, the case may include connector pins disposed within the receptacle for the remote control. Connector pins may also be disposed within the receptacles for the stimulation devices. In some instances, the case may be configured to receive two of the stimulation devices. As such, connector pins may be disposed within a receptacle for the first stimulation device and a receptacle for the second stimulation device. However, the case may be configured to receive more than or less than two of the stimulation devices and in such instances, the case may include a corresponding number of connector pins.

The case may include two of the connector pins for each of the stimulation devices and the remote control. However, the stimulation devices and the remote control may include four of the connector pads. When disposed in the receptacles, only two of the connector pads of the stimulation devices and the remote control may be engaged with the connector pins, respectively. The other two of the connection pads may be non-operational (or not used). By including four of the connector pads, however, the stimulation devices and the remote control may be disposed within the receptacles, respectively, at different orientations to permit their charging. In this manner, there may be more than one orientation by which to place stimulation devices and the remote control in the receptacles to permit their charging.

The case may also include connectors that engage with the connectors of the stimulation devices. The connectors may be disposed within the receptacles, respectively. The connectors of the case may be magnetic elements or manufactured from a ferrous material to magnetically attract to the connectors of the stimulation devices and secure the stimulation devices within the case. The receptacle for the remote control may also include magnetic elements or have a ferrous material that magnetically attracts the remote control to secure the remote control within the case.

The case may include indents, reliefs, troughs, etc., adjoined to the receptacles to permit the stimulation devices and the remote control to be easily removed (e.g., pulled) from the receptacles. The indents, for example, may accommodate a finger of the user to permit the finger of the user to lift upwards on the stimulation devices and/or the remote control to permit their removal.

The case may be formed from a housing and a lid. The lid may be adjoined or otherwise connected to the housing (e.g., via hinges, a seam, etc.). In some instances, the housing may include the receptacles for the stimulation devices and the remote control, and the lid may include the compartment. The housing and the lid may be secured together via a zipper, buckles, buttons, etc. The case may also include a port (e.g., USB-C, USB, etc.) for charging a battery of the case. The battery of the case may be communicatively connected to the connector pins in order to charge the stimulation devices and the remote control. The port may be located along an exterior of the housing.

In some instances, the user may utilize a device (e.g., mobile device, wearable, tablet, computer, etc.) to control the stimulation devices (i.e., similar to the remote control). In addition, the device may be used to set up (e.g., place) the stimulation devices. For example, the device may display a series of user interfaces (UIs) that guide the user in placing the pads on their body, the stimulation device, selecting the mode(s), and/or selecting the setting(s) by which to control the stimulation devices. The UIs may permit the user to input where on their body the pain, discomfort, etc., is located, how to apply the pads, and/or how to control the stimulation devices. The UIs may also indicate common causes of the pain, discomfort, etc., other exercises to perform to relieve the pain, discomfort, etc., and so forth.

In some instances, the case may include an RFID that is used to prompt the device to display the UIs. For example, the user may tap or place the device in proximity to the RFID. In response to detecting the RFID, or in response to data captured from the RFID, the device may display the UIs and walk the user through the setup process for placing the pads on their body. The device may include an associated RFID reader for receiving the data from the case. The device may also track and record, via an application, for example, a history of the user, their use of the stimulation device (e.g., settings, duration, etc.), and so forth.

Although described as including certain components, the stimulation device, the remote control, the pads, and/or the case may be different from those described. For example, the stimulation devices may be capable of delivering heat, vibration, and/or cooling to the user. In such instances, the remote control and/or the device may be used to control these functions.

The present disclosure provides an overall understanding of the principles of the structure, function, device, and system disclosed herein. One or more examples of the present disclosure are illustrated in the accompanying drawings. Those of ordinary skill in the art will understand and appreciate that the devices, the systems, and/or the methods specifically described herein and illustrated in the accompanying drawings are non-limiting embodiments. The features illustrated or described in connection with one embodiment or instance may be combined with the features of other embodiments or instances. Such modifications and variations are intended to be included within the scope of the disclosure and appended claims.

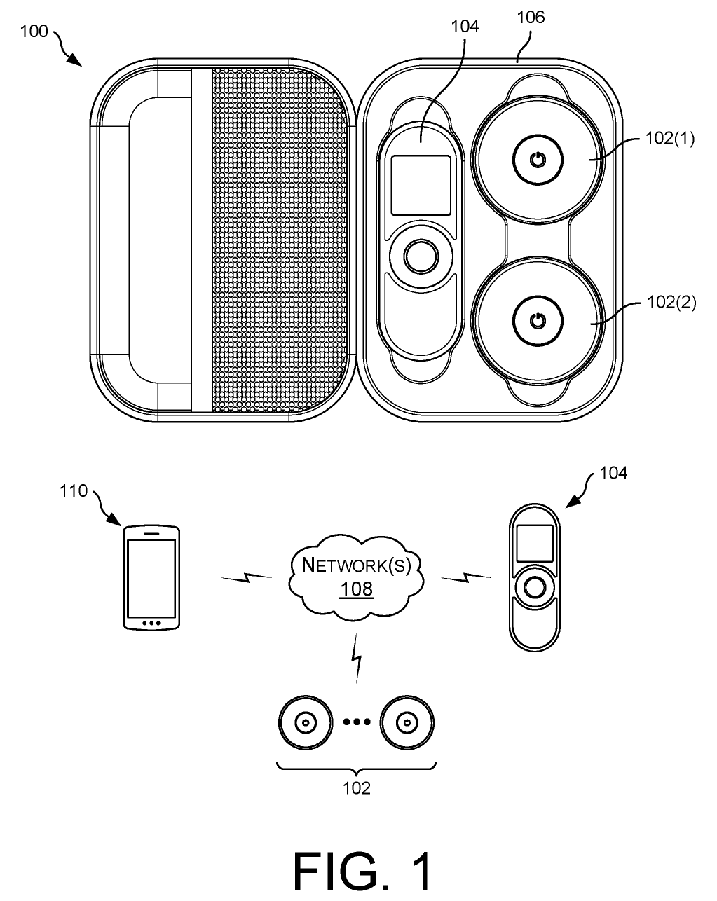

FIG. 1 illustrates an example system 100 configured to deliver electrical pulses to a user for TENS or EMS, according to an embodiment of the disclosure. In some instances, the system 100 may include stimulation devices 102, a remote control 104, and a case 106. The stimulation devices 102 and the remote control 104 may be stored in the case 106 when not in use (e.g., for charging). In some instances, the case 106 may be configured to store two of the stimulation devices 102, such as a stimulation device 102(1) and a stimulation device 102(2).

As will be explained herein, the stimulation devices 102 may be configured to deliver electrical pulses (e.g., current) to the user for TENS and/or EMS. For example, although not shown, pads may adhere to the user and the stimulation devices 102 may couple to the pads. The stimulation devices 102 may include a pulse generator that generates the electrical pulses, and the electrical pulses may be transmitted to the user via the pads. The pads may be disposed at any location, or area, on the user, such as the back, arms, legs, neck, etc.

The stimulation device 102 and the remote control 104 may be configured to communicate over one or more network(s) 108, such as Wi-Fi, Cellular, Zigbee, Bluetooth, etc. The remote control 104 may be used to control modes and/or settings of the stimulation devices 102, such as a pattern of the electrical pulses, an intensity of the electrical pulses, a duration of the electrical pulses, a frequency of the electrical pulses, and so forth. In some instances, the stimulation devices 102 may be controlled independently or dependently of one another.

The stimulation devices 102, the remote control 104, and/or the case 106 may also communicatively connect to a device 110 over the network(s) 108. The device 110 may be representative of any suitable device, such as a mobile phone, wearable, tablet, etc. In some instances, the device 110 may be used to guide the user (e.g., via an application, website, etc.) through a series of user interfaces (UIs) for setting up the stimulation devices 102. For example, the device 110 may display UIs that prompt the user to indicate areas of pain, discomfort, etc., and in response, the UIs may instruct the user as to where to place the pads, modes and/or settings of the stimulation devices 102, etc.

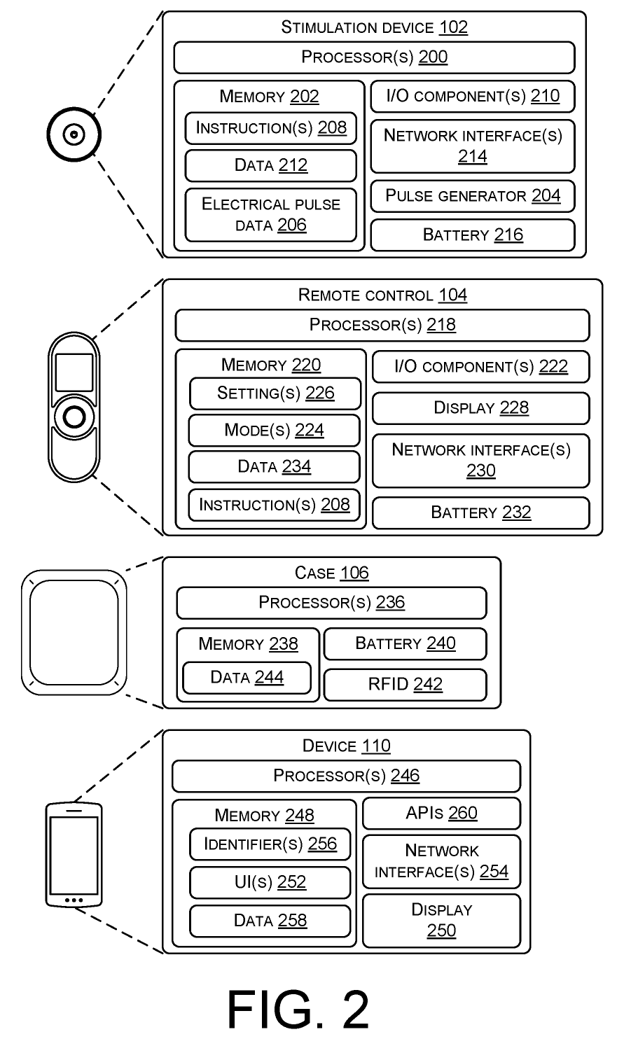

FIG. 2 illustrates select functional components of the stimulation device 102, the remote control 104, the case 106, and the device 110, according to an embodiment of the present disclosure.

The stimulation device 102 includes processor(s) 200 and memory 202, where the processor(s) 200 may perform various functions and operations associated with controlling the stimulation device 102, and the memory 202 may store instructions executable by the processor(s) 200 to perform the operations described herein. The stimulation device 102 may include a pulse generator 204 that generates electrical pulses administered to the user. In some instances, the pulse generator 204 may generate electrical pulses with currents up to 120 mA and/or voltages up to 120V. The electrical currents may also be generated at suitable frequencies (e.g., 0 and 150Hz) and/or for any duration (e.g., one minute, twenty minutes, etc.). The pulse generator 204 may generate the electrical pulses according to electrical pulse data 206, which is stored in the memory 202. The electrical pulse data 206 may indicate the timing, duration, frequency, duration, etc., by which to control the pulse generator 204. For example, based at least in part on the electrical pulse data 206, the pulse generator 204 may generate the electrical pulses.

The stimulation device 102 may be configured to receive instruction(s) 208 from the remote control 104, for example, where the instruction(s) 208 indicate instructions, commands, signals, etc., for controlling the stimulation device 102. The instruction(s) 208 may indicate or be associated with the electrical pulse data 206, or the stimulation device 102 may generate the electrical pulse data 206 based at least in part on the instruction(s) 208 received from the remote control 104. The instruction(s) 208 may indicate various modes, settings, etc., by which to operate the stimulation device 102 (e.g., duration of electrical pulses, frequency of electrical pulses, intensity of electrical pulses, etc.). Generally, in response to receiving the instruction(s) 208, the stimulation device 102 may cause the pulse generator 204 to generate the electrical pulses.

The stimulation device 102 may include I/O component(s) 210, such as a button, lighting element(s), speaker(s), etc. The I/O component(s) 210 may be used to control the stimulation device 102 and/or indicate an operational status of the stimulation device 102. For example, a button may be used to power on/off the stimulation device 102, sync or pair the stimulation device 102 with the remote control 104, and so forth. The lighting element(s) may illuminate according to the operational status, such as whether the stimulation device 102 is powered on, powering off, synced or paired with the remote control 104, etc.

The memory 202 is shown storing data 212, where the data 212 may indicate settings by which to control the I/O component(s) 210, presses of the button, whether the stimulation device 102 is connected to the pad, or has conductivity with the pad, etc. Network interface(s) 214 permit the stimulation device 102 to communicate with the remote control 104, for example, and other devices over the network(s) 108. Although described as being controlled by the remote control 104, in some instances, the stimulation device 102 may be controlled via the device 110, and/or the I/O component(s) 210 may be used to control the stimulation device 102. As such, the stimulation device 102, in some instances, may operate independently of the remote control 104. The stimulation device 102 may also include a battery 216 for powering components, such as the pulse generator 204, the network interface(s) 214, etc.

The remote control 104 includes processor(s) 218 and memory 220, where the processor(s) 218 may perform various functions and operations associated with controlling the remote control 104, and the memory 220 may store instructions executable by the processor(s) 218 to perform the operations described herein. As introduced above, the remote control 104 may be used to control the stimulation device 102. For example, the remote control 104 may include I/O component(s) 222, such as buttons, switches, toggles, etc., that allow the user to power on/off the remote control 104, select mode(s) 224, etc. The mode(s) 224 may be associated with predetermined routines, patterns, programs, etc., of the electrical pulses delivered to the user. For example, different patterns of the electrical pulses, in duration, frequency, and intensity, may be administered based at least in part on the mode(s) 224. Examples of the mode(s) 224 may include massaging, pain relief, stretch, deep tissue, etc. The mode(s) 224 may be indicative of the type of TENS or EMS treatment to be delivered to the user. The stimulation device 102 may be controlled according to any number of mode(s) 224, such as one, two, five, ten, etc. The user may utilize the I/O component(s) 222 to select and scroll through the mode(s) 224. In addition, the user may toggle between the mode(s) 224, switch between the mode(s) 224, etc.

The mode(s) 224 may indicate, or be associated with, setting(s) 226, where the setting(s) 226 indicate the duration, frequency, and intensity of the electrical pulses. For example, for each of the mode(s) 224, the setting(s) 226 may indicate a frequency of the electrical pulses, a duration of the electrical pulses, an intensity of the electrical pulses, and so forth. The mode(s) 224 and the setting(s) 226 may be used to generate the instruction(s) 208 that are sent to the stimulation device 102. For example, the instruction(s) 208 may indicate the mode(s) 224 and/or the setting(s) 226, which, upon receipt, may be used by the pulse generator 204 to generate the electrical pulses.

The remote control 104 includes a display 228 that displays information associated with the mode(s) 224 and/or the setting(s) 226. For example, the display 228 may display information of the selected mode, an intensity of the electrical pulses delivered by the pulse generator 204, an amount of time remaining on the mode 224, or the electrical pulses. The I/O component(s) 222 may also be used to pause the delivery of the electrical pulses, which may correspondingly be displayed on the display 228. For example, the user may pause or switch between the mode(s) 224. Regardless, in response to input(s) at the I/O component(s) 222, the remote control 104 may communicate with the stimulation device 102 for adjusting, pausing, resuming, etc., the electrical pulses being administered. In some instances, the remote control 104 may continuously communicate with the stimulation devices 102, or may communicate with the stimulation devices 102 only after receiving input(s) associated with controlling the stimulation device(s) 102. The display 228 may also display other information, such as a time, a power level (e.g., charge) of the stimulation devices 102, the remote control 104, etc.

In some instances, the display 228 may operate according to different states (e.g., modes, functions, etc.). For example, after selecting a mode 224 and communicating the instruction(s) 208 to the stimulation devices 102, the remote control 104 may go into a sleep mode whereby the display 228 may turn off. However, the stimulation device 102 may still operate according to the instruction(s) 208 to deliver the electrical pulses. Upon receiving input at the I/O component(s) 222, the display 228 may wake up and illuminate to display information.

The remote control 104 may be used to control any number of the stimulation devices 102, such as one, two, three, etc. In some instances, the stimulation devices 102 may be independently or dependently controlled. In such instances, the remote control 104 may coordinate the operation of the stimulation devices 102 with one another, or may communicate with the stimulation devices 102 separately. The display 228 may display information associated with each of the stimulation devices 102, for example, indicating the intensity of their electrical pulses, the mode 224, etc. The remote control 104 may store indicators, identifiers, etc., of the stimulation devices 102 to which its connected. The remote control 104 may include network interface(s) 230 for communicating over the network(s) 108. The remote control 104 may also communicate with the device 110, for example, to send data associated with the electrical pulses being delivered.

The remote control 104 may also include a battery 232 for powering components of the remote control 104, such as the display 228. The memory 220 is shown storing data 234, where the data 234 may indicate input(s) at the I/O component(s) 222, etc. The data 234 may also indicate whether the stimulation device 102 is connected to the pad, or more generally, the user. For example, the instruction(s) 208 may be sent to the stimulation devices 102 based on the stimulation devices 102 being connected to the pad, a conductivity between the electrodes on the pad, and so forth. Stated alternatively, the electrical pulses may be administered only when the stimulation device 102 is connected to the pad and/or when conductivity exists between connectors on the pad. If the pad becomes disconnected from the user during use, or if the stimulation device 102 becomes disconnected from the pad, the electrical pulses may cease to be output. In such instances, the remote control 104 may receive the data 234 indicative of such, and in response, cease transmission of the instruction(s) 208, pause the mode 224 and/or electrical pulses, and/or update information displayed on the display 228. In addition, the stimulation device 102 itself may determine whether a connection has been interrupted (e.g., lost, lack, disengaged, etc.), and in response, may cease generation of the electrical pulses. In part, this is due to the circuit being interrupted between the electrodes of the pad or the connectors of the pad and the stimulation device 102.

The case 106 includes processor(s) 236 and memory 238, where the processor(s) 236 may perform various functions and operations associated with controlling the case 106, and the memory 238 may store instructions executable by the processor(s) 236 to perform the operations described herein. The case 106 may include a battery 240 for powering, or charging, the battery 216 of the stimulation devices 102 and the battery 232 of the remote control 104. The battery 240 may be charged via mains power, or from other batteries.

The case 106 includes an RFID 242 that may be used in conjunction with the device 110 to display a series of UIs to assist the user in setting up the stimulation devices 102 and/or controlling the stimulation devices 102. For example, the device 110 may be tapped on an area of the case 106 and/or brought in to proximity of the RFID 242 in the case 106. In response, the device 110 may display a website, link, series of UIs, etc. Generally, the UIs may allow the user to input areas of pain, discomfort, etc., and in response, the UIs may display how to apply the pads to their body, the types of pads to use on their body, how to control the stimulation devices 102 (e.g., the mode(s) 224, the setting(s) 226, etc.), and so forth.

The case 106 may also store data 244, which may indicate whether the stimulation devices 102 are disposed in the case 106, whether the remote control 104 is disposed in the case 106, and so forth. For example, proximity sensor(s) may be disposed in the case 106, and may be used to detect the presence of the stimulation devices 102 and the remote control 104, respectively. If present, or connected to the connectors, the case 106 may permit power to be transferred to the stimulation devices 102 and the remote control 104. The data 244 may also indicate a charge of the battery 240, which may be displayed on the display 228 of the remote control 104. Additionally, or alternatively, the case 106 may include I/O component(s) that indicate the charge of the battery 240.

The device 110 includes processor(s) 246 and memory 248, where the processor(s) 246 may perform various functions and operations associated with controlling the device 110, and the memory 248 may store instructions executable by the processor(s) 246 to perform the operations described herein. The device 110 may include a display 250 for displaying UIs 252 to the user. For example, in response to receiving data 258 from the user (e.g., touch input, areas of pain or discomfort, feedback, etc.), the UIs 252 may be displayed. As indicated above, the UIs 252 may guide the user through placing the pads on their body, how to control the stimulation devices 102, and so forth. In some instances, the device 110 may store or operate an application that displays the UIs 252. The application may also record a history of the user, previous therapy provided via stimulation devices 102, and so forth. The user may also provide feedback via the device 110, such as whether the TENS and/or EMS provided relief, whether pain and/or discomfort is present, etc. Such feedback may be utilized in the future to provide subsequent TENS and/or EMS. Such data may be stored within a profile of the user.

The device 110 may include APIs 260 for communicating with other components, applications, etc., on the device 110 (e.g., camera, the display 250, etc.). The device 110 also includes network interface(s) 254 for communicating with the case 106, the stimulation devices 102, the remote control 104, etc. In some instances, although the remote control 104 is described as controlling the stimulation devices 102, the device 110 may be used to control the stimulation devices 102. In such instances, the device 110 may communicate with the stimulation devices 102 via the network(s) 108. The device 110 may store identifier(s) 256 of the case 106, the remote control 104, the stimulation devices 102, etc.

Although certain components of the stimulation device 102, the remote control 104, the case 106, and the device 110 are shown, the stimulation device 102, the remote control 104, the case 106, and/or the device 110 may include additional or alternative components. For example, the stimulation device 102 may include heating elements, motors, etc., that provide heating, vibration, etc., to the user. Moreover, as another example, the I/O component(s) 222 may include speaker(s) to output sound associated with the electrical pulses being delivered.

As used herein, a processor, such as the processor(s) 200, the processor(s) 218, the processor(s) 236, and/or the processor(s) 246 may include multiple processors and/or a processor having multiple cores. Further, the processor(s) 200, the processor(s) 218, the processor(s) 236, and/or the processor(s) 246 may comprise one or more cores of different types. For example, the processor(s) 200, the processor(s) 218, the processor(s) 236, and/or the processor(s) 246 may include application processor units, graphic processing units, and so forth. In one implementation, the processor(s) 200, the processor(s) 218, the processor(s) 236, and/or the processor(s) 246 may comprise a microcontroller and/or a microprocessor. The processor(s) 200, the processor(s) 218, the processor(s) 236, and/or the processor(s) 246 may include a graphics processing unit (GPU), a microprocessor, a digital signal processor or other processing units or components known in the art. Alternatively, or in addition, the functionally described herein can be performed, at least in part, by one or more hardware logic components. For example, and without limitation, illustrative types of hardware logic components that may be used include field-programmable gate arrays (FPGAs), application-specific integrated circuits (ASICs), application-specific standard products (ASSPs), system-on-a-chip systems (SOCs), complex programmable logic devices (CPLDs), etc. Additionally, each of the processor(s) 200, the processor(s) 218, the processor(s) 236, and/or the processor(s) 246 may possess its own local memory, which also may store program components, program data, and/or one or more operating systems.

Memory, such as the memory 202, the memory 220, the memory 238, and/or the memory 248 may include volatile and nonvolatile memory, removable and non-removable media implemented in any method or technology for storage of information, such as computer-readable instructions, data structures, program component, or other data. Such memory may include, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, RAID storage systems, or any other medium which can be used to store the desired information and which can be accessed by a computing device. The memory 202, the memory 220, the memory 238, and/or the memory 248 may be implemented as computer-readable storage media (“CRSM”), which may be any available physical media accessible by the processor(s) to execute instructions stored on the memory. In one basic implementation, CRSM may include random access memory (“RAM”) and Flash memory. In other implementations, CRSM may include, but is not limited to, read-only memory (“ROM”), electrically erasable programmable read-only memory (“EEPROM”), or any other tangible medium which can be used to store the desired information and which can be accessed by the memory 202, the memory 220, the memory 238, and/or the memory 248. The memory 202, the memory 220, the memory 238, and/or the memory 248 is an example of non-transitory computer-readable media. The memory 202, the memory 220, the memory 238, and/or the memory 248 may store an operating system and one or more software applications, instructions, programs, and/or data to implement the methods described herein and the functions attributed to the various systems.

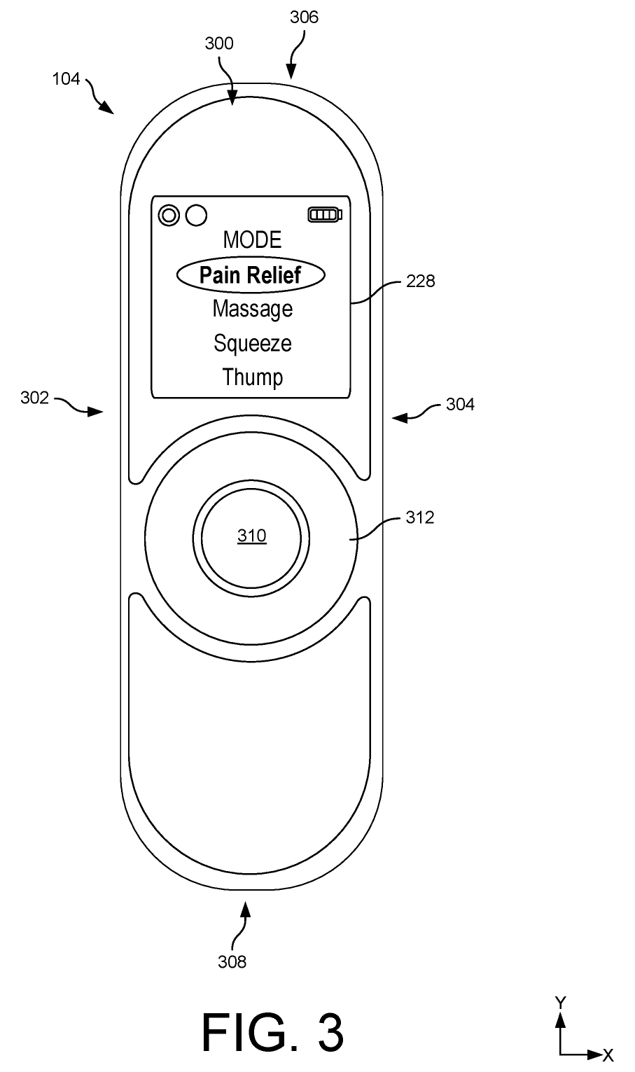

FIG. 3 illustrates a top view of the remote control 104, according to an embodiment of the present disclosure. The remote control 104 may include a top 300, a bottom (e.g., opposite the top 300, spaced apart in the Z-direction), a first side 302, a second side 304 opposite the second side 304 (e.g., spaced apart in the X-direction), a first end 306, and a second end 308 opposite the first end 306 (e.g., spaced apart in the Y-direction). The top 300 may include the display 228, a first button 310, and a second button 312.

The display 228 may output information associated with the mode(s) 224 and/or the setting(s) 226. For example, the display 228 may output a selected mode of the mode(s) 224, a timing or duration of the electrical pulses being administered in accordance with the mode(s) 224, and so forth. The display 228 may also display information associated with the number of the stimulation devices 102 connected to the remote control 104, a charge of the stimulation devices 102, a charge of the remote control 104, and so forth. The display 228 may also present additional information, such as the intensity of the electrical pulses being administered.

The first button 310 may correspond to an on/off button of the remote control 104, a select button, etc. The user may press and/or hold the first button 310 to power on/off the remote control 104. After powering on the remote control 104, the first button 310 may be used to select one of the mode(s) 224 (e.g., via toggling between the mode(s) 224 on the display 228). The first button 310 may also be used to pause the electrical pulses, for example, after the stimulation devices 102 have been delivering the electrical pulses. The second button 312 may be disposed around the first button 310. The second button 312 may be used by the user to toggle between different modes, adjust the setting(s) 226 of the mode(s) 224 (e.g., increase intensity, increase timing, increase frequency, etc.). The second button 312 may also be used to toggle between different screens or displays on the display 228.

As shown, the display 228 may be located between the second button 312 and the first end 306. In some instances, the first button 310 may be disposed centrally between the first side 302 and the second side 304, and/or centrally between the first end 306 and the second end 308. Although a particular shape and/or arrangement of the display 228, the first button 310, and/or the second button 312 is shown, other various arrangements are envisioned. For example, the first button 310 and/or the second button 312 may be located and/or shaped differently than shown, and/or the remote control 104 may include more than or less than two of the buttons. Moreover, the display 228 may be a touch-sensitive display. The remote control 104 may also include other I/O component(s) 222.

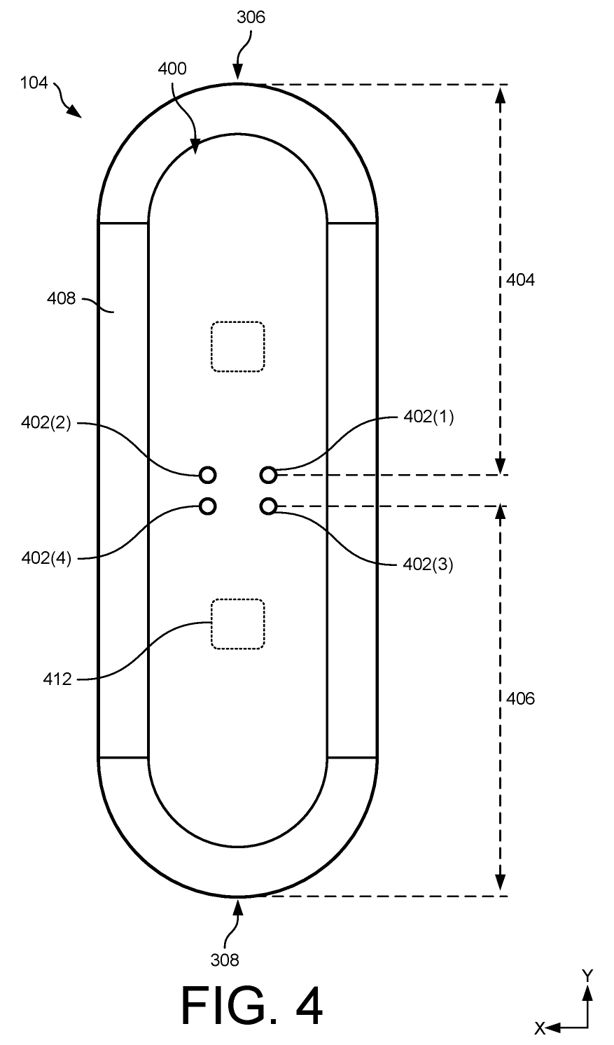

FIG. 4 illustrates a bottom view of the remote control 104, according to an embodiment of the present disclosure. The remote control 104 includes a bottom 400 spaced apart from the top 300 (e.g., in the Y-direction). The bottom 400 includes connector pads 402 that are configured to engage with connector pins disposed on, within, or in the case 106. For example, when the remote control 104 resides within the case 106, for charging or storage, the connector pads 402 may engage with the connector pins of the case 106.

As shown, the remote control 104 may include four of the connector pads 402, such as connector pad 402(1), a connector pad 402(2), a connector pad 402(3), and a connector pad 402(4). The connector pins of the case 106 may engage with the connector pad 402(1) and the connector pad 402(2), or the connector pad 402(3) and the connector pad 402(4). For example, depending upon an orientation of the remote control 104 in the case 106, the connector pad 402(1) and the connector pad 402(2), or the connector pad 402(3) and the connector pad 402(4), may engage with the connector pins in the case 106. The redundant nature of the connector pads 402 permits the battery 232 of the remote control 104 to be charged regardless of the orientation of the remote control 104 in the case 106. The connector pad 402(1) and the connector pad 402(2) may be spaced apart from the first end 306 by a first distance 404. The connector pad 402(3) and the connector pad 402(4) may be spaced apart from the second end 308 by a second distance 406, which may be the same as the first distance 404.

The remote control 104 may include a housing 408. The remote control 104 may be formed from more than one of the housings 408, which may be coupled (e.g., snapped, fastened, etc.) together. The connector pads 402 may be exposed through the housing 408. As shown, the housing 408 may include a generally rectangular shape, however, other shapes are envisioned.

Magnetic elements 412 may be disposed beneath the bottom 400. The magnetic elements 412 are shown in dashed lines to indicate their position beneath the bottom 400. The magnetic elements 412 may engage (e.g., interact) with magnetic elements or a ferrous material in the case 106 to secure the remote control with the case 106. Although described as magnetic elements, alternatively, the remote control 104 may include ferrous material that engages with magnetic elements in the case 106. The remote control 104 may include more than or less than two of the magnetic elements 412.

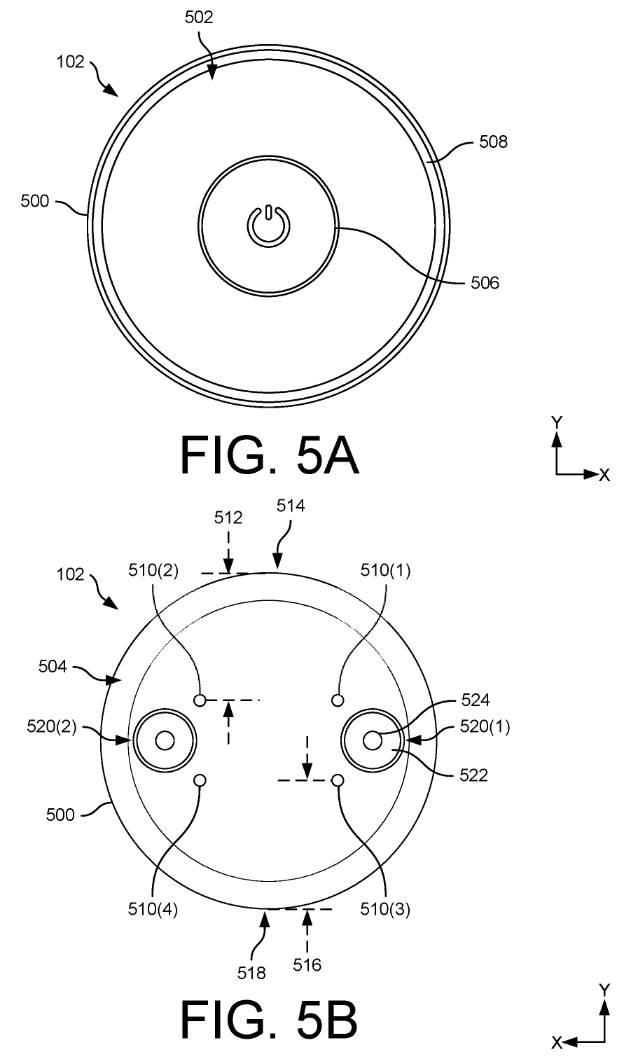

FIGS. 5A and 5B illustrate top and bottom views of the stimulation device 102, respectively, according to an embodiment of the present disclosure. The stimulation device 102 may include a housing 500 that defines a circular shape of the stimulation device 102. However, the housing 500 or the stimulation device 102 may take other shapes (e.g., square, hexagonal, etc.). In some instances, the housing 500 may be manufactured from multiple housings that are coupled together.

The stimulation device 102 may include a top 502 and a bottom 504 spaced apart from the top 502 (e.g., in the Y-direction). The top 502 may include the button 506, for example, that corresponds to an on/off button of the stimulation device 102, a sync button of the stimulation device 102, etc. A light ring 508 may be disposed on the top 502 and may be disposed around the button 506. The light ring 508 may output light (e.g., via lighting elements disposed beneath the light ring 508) according to an operational state, status, etc., of the stimulation device 102. For example, the light ring 508 may output light according to different patterns, sequences, colors, etc., based on the operation state, status, etc., of the stimulation device 102. For example, before being paired, the light ring 508 may output light in a first pattern (e.g., blinking), and after being paired, the light ring 508 may output light in a second pattern (e.g., circular). The light ring 508 may also output light based on the electrical pulses being delivered (e.g., solid).

The bottom 504 of the stimulation device 102 may include connector pads 510 that are configured to engage with connector pins disposed on, within, or in the case 106. For example, when the stimulation device 102 resides within the case 106, for charging or storage, the connector pads 510 may engage with the connector pins of the case 106. As shown, the stimulation device 102 may include four of the connector pads 510, such as connector pad 510(1), a connector pad 510(2), a connector pad 510(3), and a connector pad 510(4). The connector pins of the case 106 may engage with the connector pad 510(1) and the connector pad 510(2), or the connector pad 510(3) and the connector pad 510(4). For example, depending upon an orientation of the stimulation device 102 in the case 106, the connector pad 510(1) and the connector pad 510(2), or the connector pad 510(3) and the connector pad 510(4), may engage with the connector pins in the case 106. The redundant nature of the connector pads 510 permits the battery 216 of the stimulation device 102 to be charged regardless of the orientation of the stimulation device 102 in the case 106. The connector pad 510(1) and the connector pad 510(2) may be spaced apart from a first end 514 of the stimulation device 102 by a first distance 512. The connector pad 510(3) and the connector pad 510(4) may be spaced apart from a second end 518 by a second distance 516, which may be the same as the first distance 512.

The bottom 504 may also include connectors 520 (e.g., terminals, pads, couplers, etc.), such as a connector 520(1) and a connector 520(2). The connectors 520 may connect (e.g., secure) the stimulation device 102 within the case 106 by engaging with corresponding connectors. The connectors 520 also serve to connect the stimulation device 102 to the pads adhered to the user. In some instances, the connectors 520 may include magnetic elements or a ferrous material to engage with the connectors on the pads and the case 106, respectively. When connected to the connectors on the pads, the electrical current may be delivered to pads via connectors 520. In some instances, the connectors 520 may be disposed external to the connector pads 510.

The connectors 520 may include a flanged portion 522 and a receptacle portion 524. The flanged portion 522 may be disposed around the receptacle portion 524. The receptacle portion 524 may receive a knob (e.g., protrusion, pillar, etc.) of the pads and the case 106 (e.g., a male/female connection) to seat the stimulation device 102 on the pad or within the case 106, respectively. The flanged portion 522 and the receptacle portion 524 may include a magnetic element or may be made from a ferrous material. Moreover, when connected to the pad, for example, electrical current may flow through the flanged portion 522 and/or the receptacle portion 524 to the connectors of the pad.

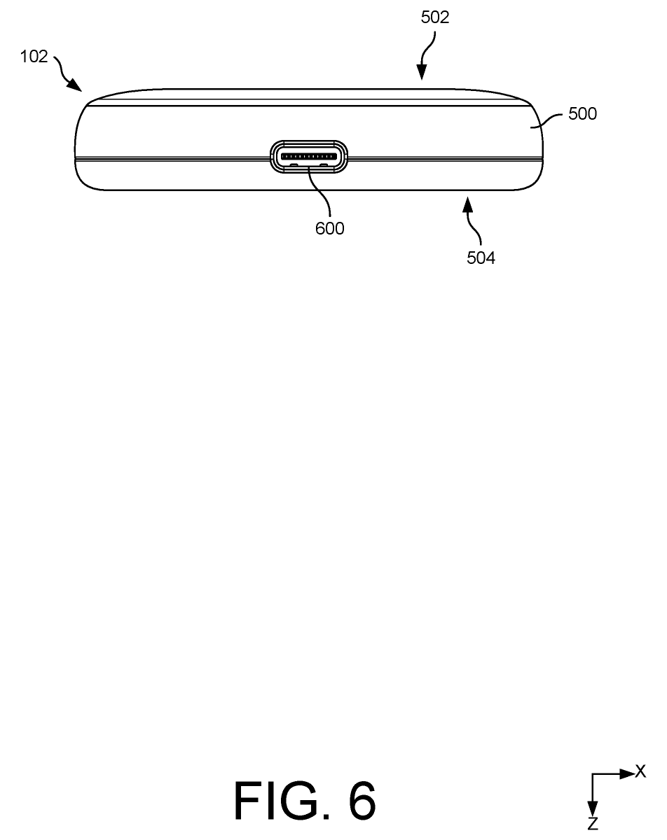

FIG. 6 illustrates an end view of the stimulation device 102, such as the second end 518, according to an embodiment of the present disclosure. The housing 500 may define an aperture for accommodating a port 600. The port 600 may be a USB-C port, for example, to connect the stimulation device 102 to other stimulation devices 102, leads, wires, cables, etc. For example, a cable may extend between the port 600 and other stimulation devices 102, leads, etc., to form a wired connection. Other ports, such as a micro-USB, are envisioned. The port 600 may also be used to charge the battery 216, independently of the case 106.

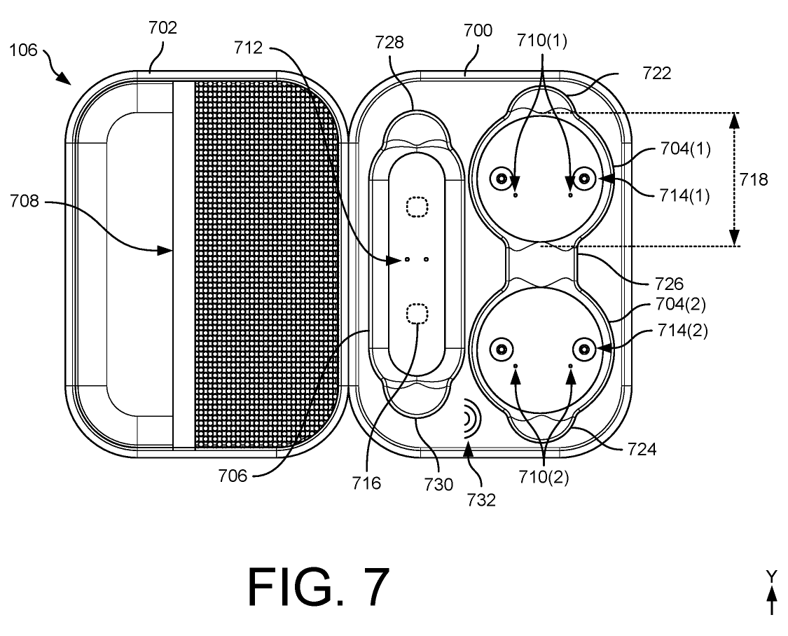

FIG. 7 illustrates the case 106, according to an embodiment of the present disclosure. The view of the case 106 in FIG. 7 illustrates the case 106 in an open state with the remote control 104 and the stimulation devices 102 removed.

The case 106 may include a housing 700 and a lid 702 coupled to the housing 700 (e.g., via hinges, seams, etc.). The case 106 is configured to store the stimulation devices 102, the remote control 104, the pads, etc., when not in use. In some instances, the case 106 includes receptacles 704 for the stimulation devices 102, a receptacle 706 for the remote control 104, and/or a compartment 708 for the pads. The case 106 includes a receptacle 704(1) for the stimulation device 102(1) and a receptacle 704(2) for the stimulation device 102(2). The receptacles 704 and the receptacle 706 may be complimentary shaped for the stimulation device 102 and the remote control 104, respectively. The compartment 708 may be used to store the pads and may include, or be formed from, a webbing or other material behind which the pads are stored.

The case 106 includes connector pins (e.g., prongs, etc.), which may be disposed within the receptacles 704 and the receptacle 706 for charging the stimulation devices 102 and the remote control 104, respectively. For example, the case 106 may include connector pins 710(1) disposed within the receptacle 704(1) for the stimulation device 102(1), connector pins 710(2) disposed within the receptacle 704(2) for the stimulation device 102(2), and connector pins 712 disposed within the receptacle 706 for the remote control 104.

The case 106 may include two of the connector pins for each of the stimulation devices 102 and the remote control 104. However, as noted above, the stimulation devices 102 may include four of the connector pads 510 and the remote control 104 may include four of the connector pads 402. When disposed in the receptacles 704 or the receptacle 706, only two of the connector pads 510 and two of the connector pads 402 may be engaged with the connector pins 710 and the connector pins 712, respectively. The other two may be non-operational (or not used). However, by including four of the connector pads 510 and four of the connector pads 402, however, the stimulation devices 102 and the remote control 104 may be disposed within the receptacles 704 and the receptacle 706, respectively, at different orientations.

The case 106 may also include connectors 714 that engage with the connectors 520 of the stimulation devices 102. The connectors 714 may be disposed within the receptacles 704, respectively. For example, connectors 714(1) may be disposed within the receptacle 704(1) and connectors 714(2) may be disposed within the receptacle 704(2). The connectors 714 may be magnetic elements or manufactured from a ferrous material to magnetically attract to the connectors 520 of the stimulation devices 102 and secure the stimulation devices 102 within the case 106.

Moreover, the receptacle 706 for the remote control 104 may also include magnetic elements 716, or have a ferrous material that magnetically attracts to the magnetic elements 412 of the remote control 104. The magnetic elements 716 within the receptacle 706 are shown in dashed lines to indicate their position beneath a surface of the receptacle 706.

The receptacle 704(1) may include a dimension 718 (e.g., width, length, etc.) that extends between diametrically opposed locations within the receptacle 704(1) (e.g., in the Y-direction). The connectors 714(1) may be disposed equidistantly between the ends of the dimension 718. In doing so, the connectors 714(1) may be centered within the receptacle 704(1) (e.g., in the Y-direction). The connector pins 710(1), however, may not be centered within the receptacle 704(1). For example, the connector pins 710(1) may be located closer to one end of the receptacle 704(1). However, having four of the connector pads 510 of the stimulation device 102 accommodates the offsetting nature of the connector pins 710(1).

Likewise, the receptacle 704(2) may include a dimension (e.g., width, length, etc.) that extends between diametrically opposed locations within the receptacle 704(2) (e.g., in the Y-direction). The dimension of the receptacle 704(2) may be the same as the dimension 718 of the receptacle 704(1). The connectors 714(2) may be disposed equidistantly between the ends of the dimension. In doing so, the connectors 714(2) may be centered within the receptacle 704(2) (e.g., in the Y-direction). The connector pins 710(2), however, may not be centered within the receptacle 704(2). For example, the connector pins 710(2) may be located closer to one end of the receptacle 704(2). However, having four of the connector pads 510 of the stimulation device 102 accommodates the offsetting nature of the connector pins 710(2).

The case 106 may include indents, reliefs, troughs, etc., adjoined to the receptacles 704 and/or the receptacle 706 to permit the stimulation devices 102 and the remote control 104 to be easily removed (e.g., pulled) from the case 106, respectively. For example, an indent 722 may disposed adjacent or connected to the receptacle 704(1), an indent 724 may be disposed adjacent or connected to the receptacle 704(2), an indent 726 may be disposed adjacent or connected to the receptacle 704(1) and the receptacle 704(2), an indent 728 may be disposed adjacent or connected to the receptacle 706, and an indent 730 may be disposed adjacent or connected to the receptacle 706. The indents may accommodate a finger of the user to permit the finger of the user to lift upwards on the stimulation devices 102 and/or the remote control 104 to permit their removal from the case 106.

The case 106 may include an icon 732 (e.g., indication, graphic, etc.) associated with an area of the case 106 corresponding to the RFID 242. For example, the icon 732 may visually indicate to the user where to tap or place the device 110. In response to the device 110 tapping or being placed in proximity to the icon 732, and correspondingly, the RFID 242, the device 110 may be caused to display the UIs 252. The icon 732 and/or the RFID 242 may be located differently than shown.

The case 106 is configured to transition to a closed state, whereby the stimulation devices 102, the remote control 104, and the pads may be secured with the case 106. For example, portions of the lid 702 and the case 106 may zip together, buckle together, etc. In some instances, and as shown, the housing 700 may define the receptacles 704 and the receptacle 706, while the lid 702 may define the compartment 708.

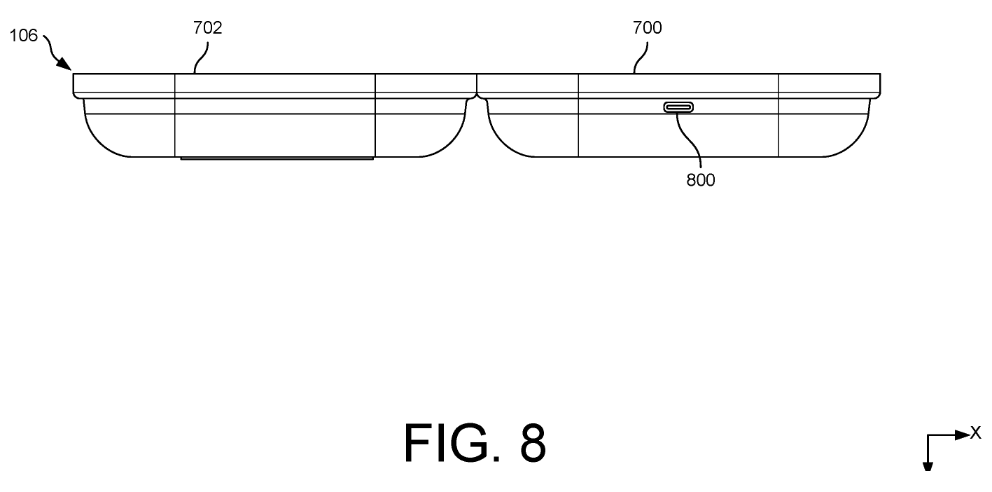

FIG. 8 illustrates a side view of the case 106, showing the case 106 in an open state, according to an embodiment of the present disclosure. Introduced above, the case 106 may include the housing 700 and the lid 702. The case 106 may include a port 800 for connecting the case 106 to mains power, for example, to charge the battery 240. The port 800 may be a USB-C connector and may be located on the housing 700 of the case 106.

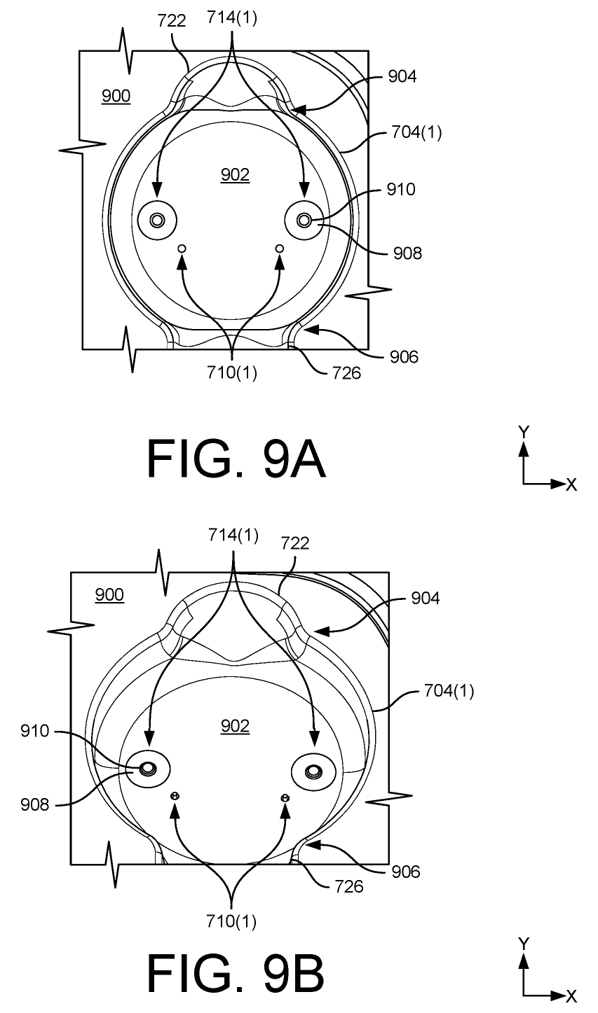

FIGS. 9A and 9B illustrate the receptacle 704(1), according to an embodiment of the present disclosure. Although the discussion in FIGS. 9A and 9B is with regard to the receptacle 704(1), the receptacle 704(2) may be similar to the receptacle 704(1).

As introduced above, the receptacle 704(1) is sized, shaped, and configured to receive the stimulation device 102, such as the stimulation device 102(1). The receptacle 704(1) may be disposed within or below a top surface 900 of the case 106. A depth of the receptacle 704(1) may receive the stimulation device 102(1). The connector pins 710(1) may be located along a bottom 902 of the receptacle 704(1). In some instances, the connector 714(1) may be centrally disposed between a first end 904 and a second end 906 of the receptacle 704(1) (e.g., diametrically opposed positions). The dimension 718 may extend between the first end 904 and the second end 906. The connectors 714(1) may be magnetic elements or ferrous materials for engaging with the connectors 520 of the stimulation device 102. The stimulation device 102(1) may snap into place within the receptacle 704(1) as the connectors 520 and the connectors 714 engage.

In some instances, the connectors 714(1) may include a flange portion 908 and a knob portion 910. The knob portion 910 may extend above the bottom 902, for engaging with the receptacle portion 524 of the connectors 520. The connectors 714(1) may include two of the connectors 714.

The connector pins 710(1) may be disposed more proximate to the second end 906 than the first end 904. However, the stimulation device 102(1) may include four of the connector pads 510, such that two of the connector pads 510 may engage with the connector pins 710(1) to accommodate different orientations of the stimulation device 102(1) in the receptacle 704(1). The connector pins 710(1) may be disposed above the bottom 902 to engage with the connector pads 510 of the stimulation device 102(1). In some instances, the connector pins 710(1) may be disposed within (e.g., in the X-direction) the connectors 714(1).

The indent 722 may be disposed adjacent to or at the first end 904. The indent 726 may be disposed adjacent to or at the second end 906. The indent 722 and the indent 726 may be adjoined to the receptacle 704(1). The indent 722 and the indent 726 may accommodate the fingers of the user.

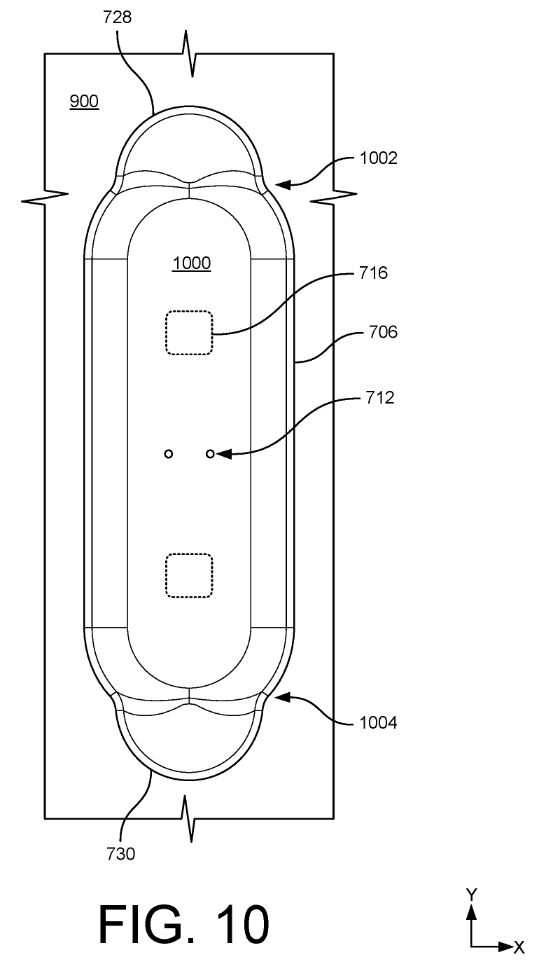

FIG. 10 illustrates the receptacle 706 of the case 106 that receives the remote control 104, according to an embodiment of the present disclosure.

The receptacle 706 is sized, shaped, and configured receive the remote control 104. The receptacle 706 may be disposed within or below the top surface 900 of the case 106. A depth of the receptacle 706 may receive the remote control 104. The connector pins 712 may be located along a bottom 1000 of the receptacle 706. In some instances, the connector pins 712 may be located closer to a first end 1002 of the receptacle 706 as compared to a second end 1004 of the receptacle 706. However, four of the connector pads 402 may accommodate a connection with the connector pins 712, whether the first end 306 of the remote control 104 is disposed adjacent to the first end 1002 or the second end 1004.

The indent 728 may be disposed adjacent to or at the first end 1002. The indent 730 may be disposed adjacent to or at the second end 1004. The indent 728 and the indent 730 may accommodate the fingers of the user.

The magnetic elements 716 may be disposed beneath the bottom 1000 to engage with the magnetic elements 412 of the remote control 104 and secure the remote control 104 within the case 106.

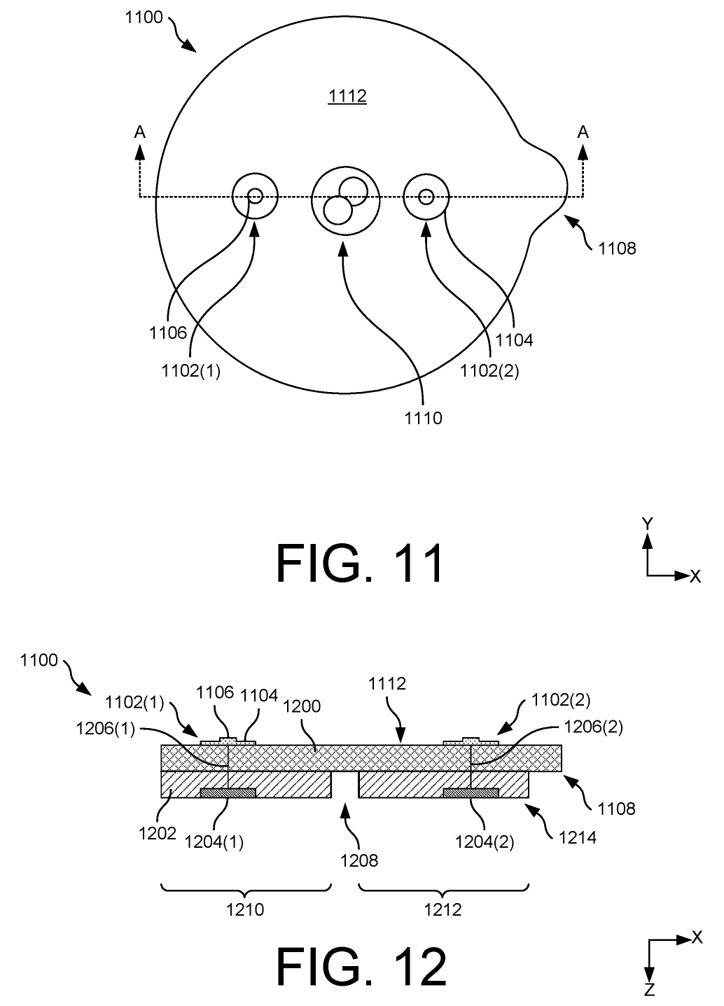

FIG. 11 illustrates an example pad 1100, according to an embodiment of the present disclosure. The pad 1100 is configured to receive the stimulation device 102, or stated alternatively, the stimulation device 102 is configured to couple to the pad 1100. The pad 1100 may include connectors 1102, such as a connector 1102(1) and a connector 1102(2). The connectors 1102 engage with the connectors 520. In some instances, the connector 520(1) engages with the connector 1102(1) and the connector 520(2) engages with the connector 1102(2). However, the connectors 1102 engage and the connectors 520 may be universal in that the connector 520(1) may engage with the connector 1102(2) and the connector 520(2) may engage with the connector 1102(1).

The connectors 1102 may include a flange portion 1104 and a knob portion 1106. The flange portion 1104 and the knob portion 1106 may be formed from the same piece of material. The knob portion 1106 may be disposed within the receptacle portion 524 during a coupling of the connectors 1102 to the connectors 520. For example, the knob portion 1106 of the connectors 1102 may be disposed within the receptacle portion 524 of the connectors 520, respectively, to seat the stimulation device 102 on the pad 1100. Given an attraction between the magnetic elements and/or the ferrous material of the stimulation device 102 on the pad 1100 may snap, click, or secure into place. However, the stimulation device 102 may be removed from the pad 1100 upon application of a sufficient amount of force.

The pad 1100 may include any shape, such as being circular. As will be discussed herein, however, other shapes, contours, etc., are envisioned. Moreover, although described as including the connectors 1102 (i.e., the connector 1102(1) and the connector 1102(2)), or one set of the connectors 1102 to receive one of the stimulation devices 102, the pad 1100 may include more than one set of the connectors 1102 for receiving more than one of the stimulation devices 102.

The connectors 1102 may be disposed above a top surface 1112 of the pad 1100. Moreover, the pad 1100 may include a tab 1108 that enables the user to grip or otherwise grasp the pad 1100 to remove the pad 1100 from a backing (e.g., prior to placing on the skin), as well as to assist the user in removing the pad 1100 from their person (e.g., after placing on the skin).

The top surface 1112 may also include an indicia 1110 (e.g., icon, lettering, symbol, etc.) associated with a type of the pad 1100. In some instances, the pad 1100 may be used at any location on the user, such as the legs, back, neck, arms, etc.

FIG. 12 illustrates a cross-sectional view of the pad 1100, taken along line A—A of FIG. 11, according to an embodiment of the present disclosure. The pad 1100 may be manufactured from one or more layers. For example, a first layer 1200 may be manufactured from a first material (e.g., neoprene, rubber, etc.) a second layer 1202 may be manufactured from a second material (e.g., conductive hydrogel, hydrocolloid, urethane, etc.) different than the first material. The second layer 1202 is configured to be disposed adjacent to, or in contact with, the skin of the user. The first layer 1200 may define the top surface 1112 above which the connectors 1102 are disposed. In addition, the first layer 1200 may define the tab 1108.

The pad 1100 may include electrodes 1204, such as an electrode 1204(1) and an electrode 1204(2), disposed at least partially within the second layer 1202. The connectors 1102 may be electrically coupled, for example, via wires 1206, to the electrodes 1204. For example, the connector 1102(1) may be electrically coupled to the electrode 1204(1) via a wire 1206(1), and the connector 1102(2) may be electrically coupled to the electrode 1204(2) via a wire 1206(2). The second layer 1202 may include a void 1208 (e.g., channel, break, etc.) that represents a disconnect between the electrodes 1204. The void 1208 may separate a first area 1210 and a second area 1212 of the second layer 1202, where the electrode 1204(1) is disposed within the first area 1210 and the electrode 1204(2) is disposed within the second area 1212. The size of the first area 1210 and/or the second area 1212 may be the same, and/or dependent upon the size of the pad 1100. Given that the second layer 1202 is manufactured from a conductive material, and the first area 1210 and the second area 1212 are conductive, the void 1208 accommodates the differently charged electrodes.

When adhered to the user, the electrical pulses (i.e., the electrical current) may flow from the electrode 1204(1) to the electrode 1204(2), or vice versa. In this sense, the user (or their skin) may complete a circuit to allow current to flow between the electrodes 1204. However, if the pad 1100 is removed from the user or one of the electrodes 1204 loses contact with the user, conductivity is broken and the electrical pulses may be ceased. The second layer 1202 may be manufactured from a conductive material that permits the electrical pulses to be transferred to the user. In some instances, the electrodes 1204 may be disposed within (e.g., spaced apart) or along (e.g., flush) with a bottom surface 1214 of the pad 1100.

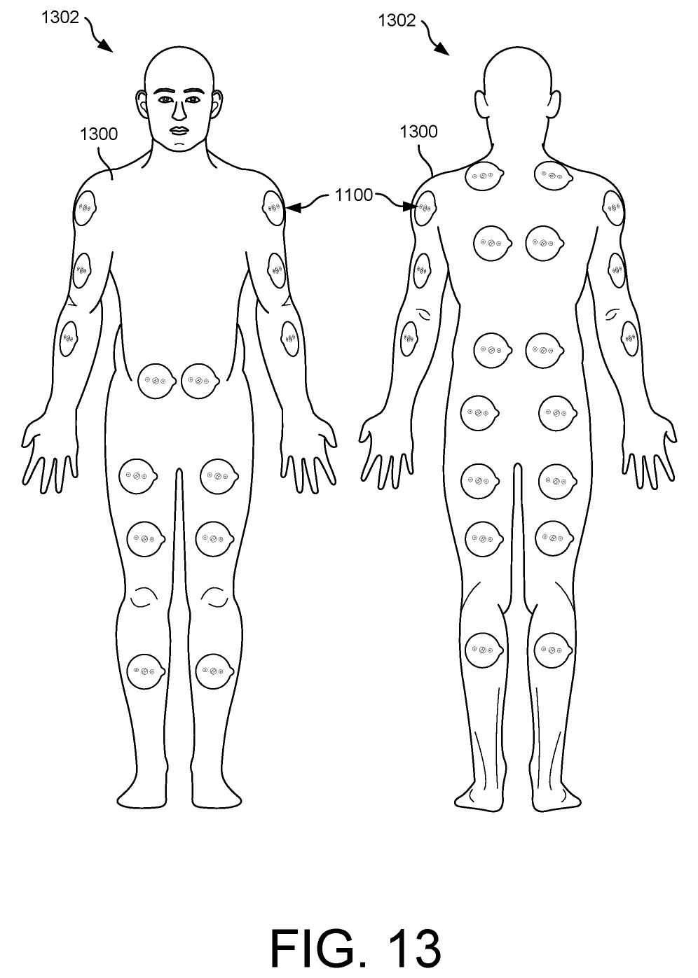

FIG. 13 illustrates an example placement of the pad 1100 on a body 1300 of a user 1302, according to an embodiment of the present disclosure. The pad 1100, as introduced above, may be a first type of pad that represents a spot or trigger point pad, whereby the pad 1100 may be placed at a plurality of locations. For example, the pad 1100 may be placed along the shoulders, back, arms, legs, abdomen, thigh, buttocks, etc. The placement of the pad 1100 is exemplary, and the pad 1100 may be placed at additional or alternative locations depending upon the relief, discomfort, etc., of the user 1302.

In some instances, more than one of the pads 1100 may be used in unison. For example, the remote control 104 may connect to more than one of the pads 1100, such as two, three, four, etc., and the remote control 104 may be used to individually or collectively control the pads 1100. As an example, a first pad may be placed on the right-side of the upper back of the user 1302 and a second pad may be placed on the left-side of the upper back of the user 1302. Here, the user 1302 may operate the first pad and the second pad collectively for TENS or EMS. Alternatively, a first pad may be placed on the left thigh of the user 1302 and a second pad may be placed on the right arm of the user. The user 1302 may operate the first pad and the second pad individually, or independently of one another, for TENS or EMS.

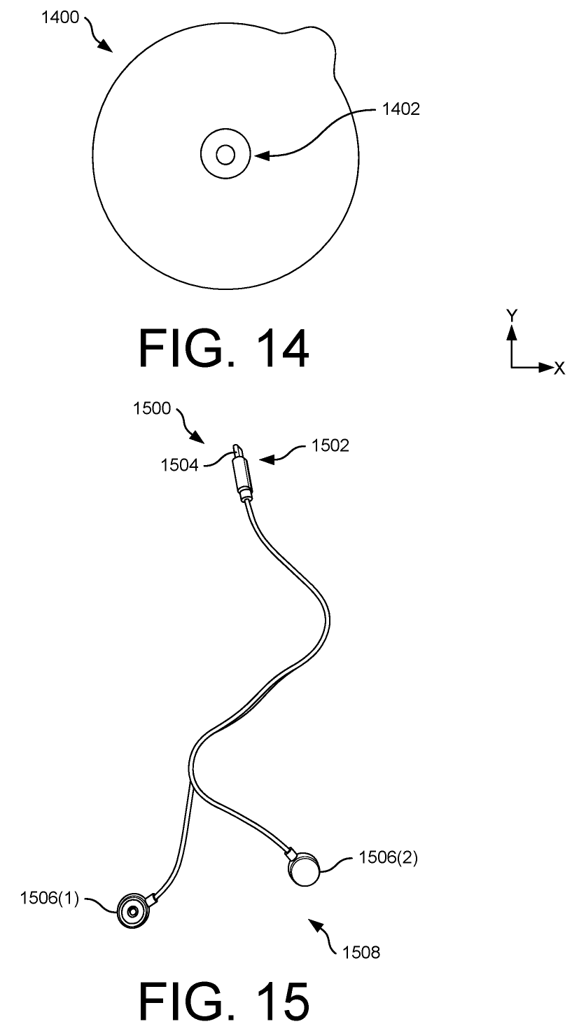

FIG. 14 illustrates an example pad 1400 that may be used in conjunction with the pad 1100 (or other pads), according to an embodiment of the present disclosure. The pad 1400 includes a connector 1402. Compared to the pad 1100, the pad 1400 may only include one connector (i.e., a single connector). The connector 1402 itself may be similar to one of the connectors 1102. The pad 1400 may connect to the stimulation device 102 via a cable, wire, etc. For example, the cable may be disposed in the port 600 and may include an end that couples to the connector 1402.

The pad 1400 may include a single electrode connected to the connector. To create a circuit, the pad 1400 may be used in conjunction with another of the pads 1400. In this manner, the pads 1400 may be connected to the stimulation device 102 via the port 600. However, a first of the pads 1400 may be placed at a first location on the user and a second of the pads 1400 may be placed at a second location on the user that is spaced apart from the first of the pads 1400. The spacing between the pads 1400 may be limited to a length of the cable. However, given that the two of pads 1400 are in contact with the user, current may flow from the electrode on the first of the pads 1400 to the electrode on the second of the pads 1400, and vice versa.

The electrical pulses delivered by the pads 1400 may be complementary or the same as the electrical pulses delivered via the stimulation device 102. In this manner, the pulse generator 204 of the stimulation device 102 may deliver the electrical pulses to the user via the pad 1100, for example, and the pads 1400. The pads 1400 may not possess their own pulse generator but may receive the electrical pulses (or signals) from the stimulation device 102.

FIG. 15 illustrates an example cable 1500 that may be used to connect the pads 1400 to the stimulation device 102, according to an embodiment of the present disclosure. The cable 1500 may include a first end 1502 having a connector 1504 configured to be disposed within the port 600. The cable 1500 may split and include multiple of the connectors 1506 at a second end 1508. A connector 1506(1) may couple to a first of the pads 1400 and a connector 1506(2) may connect to a second of the pads 1400. The connectors 1506 themselves may be similar to the connectors 520 of the stimulation device 102, for example, for connecting to the connectors 1402, respectively.

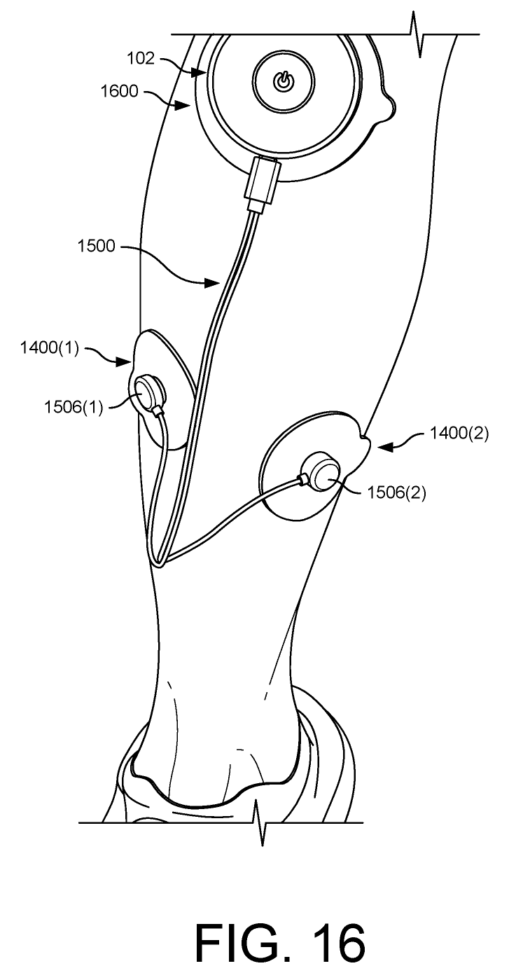

FIG. 16 illustrates an example use of the pads 1400 in conjunction with the stimulation device 102, according to an embodiment of the present disclosure. The stimulation device 102 may be connected to a pad 1600 (e.g., the pad 1100), which is adhered to a leg of the user. Moreover, the pads 1400 may be adhered to the user and connected to the stimulation device 102 via the cable 1500. For example, a pad 1400(1) may connect to the stimulation device 102 via the connector 1506(1) and a pad 1400(2) may connect to the stimulation device 102 via the connector 1506(2). During operation, the stimulation device 102 may deliver electrical pulses to the user via the electrodes 1204 on the bottom of the pad 1100. Moreover, the stimulation device 102 may deliver electrical pulses to the user via electrodes on the bottom of the pads 1400, respectively. More specifically, current may flow between the electrode on the pad 1400(1) and the electrode on the pad 1400(2). This completes a circuit between the electrodes of the pads 1400 and permits electrical pulses to be administered to the user.

Although the stimulation device 102 is shown and described as connecting to two of the pads 1400, the stimulation device 102 may connect to more than or less than two of the pads 1400. In such instances, the stimulation device 102 may include more than one of the ports 600, and/or the cable 1500 may include more than two of the connectors 1506. Additionally, or alternatively, rather than connecting to the pads 1400, the stimulation device 102 may connect to other stimulation devices 102, for example, via a cable.

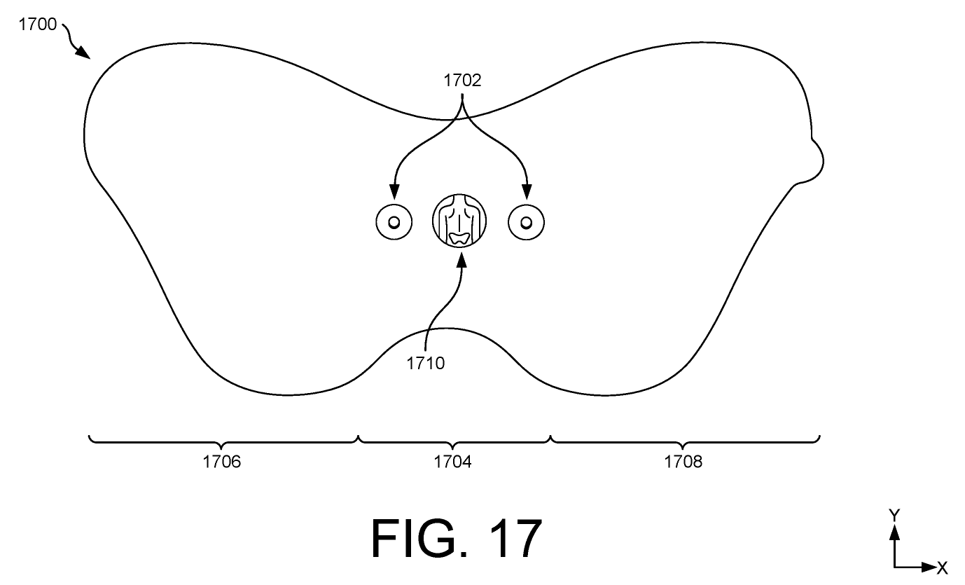

FIG. 17 illustrates an example pad 1700, according to an embodiment of the present disclosure. In some respects, the pad 1700 may be similar to the pad 1100. For example, the pad 1700 may include connectors 1702 for connecting to the connectors 520 of the stimulation device 102. As shown, the pad 1700 may include a different shape and/or may be a different type of pad than the pad 1100. In some instances, the pad 1700 may be used for a lower back of the user 1302. The pad 1700 may include a central portion 1704, a first wing 1706 that extends from the central portion 1704, and a second wing 1708 that extends from the central portion 1704. The connectors 1702 may be located on the central portion 1704. The shape of the pad 1700 accommodates a shape or contour of the lower back of the user 1302. An indicia 1710 may be disposed on the pad 1700 to indicate the type of the pad.

Like the pad 1100, the pad 1700 may include electrodes on a bottom to deliver the electrical pulses to a user. The areas of the pad 1700 may be separate by a void, and the areas may be different (e.g., larger) than the first area 1210 and/or the second area 1212.

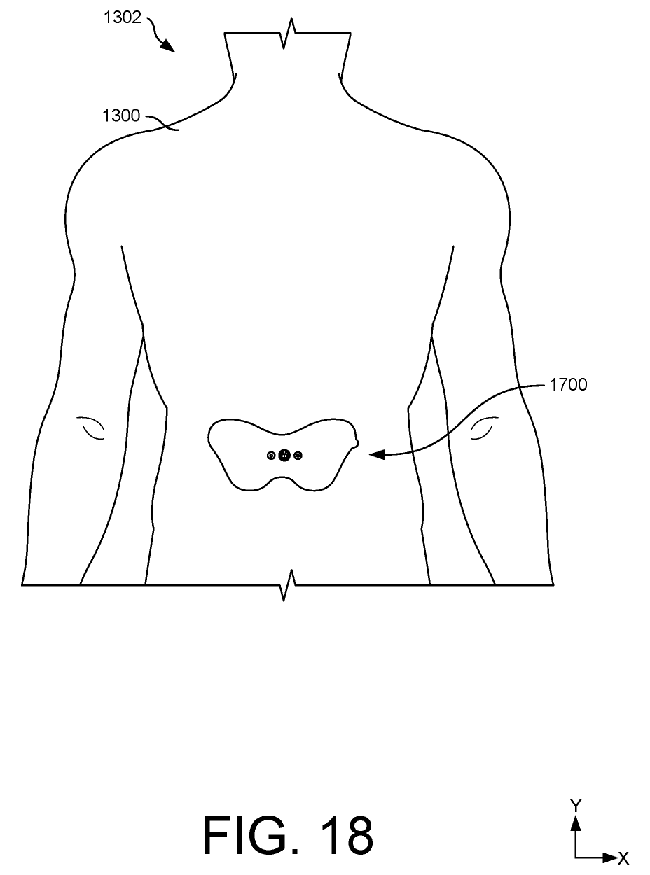

FIG. 18 illustrates the pad 1700 applied to the body 1300 of the user 1302, according to an embodiment of the present disclosure. As introduced above, the pad 1700 may be applied to the lower back of the user 1302.

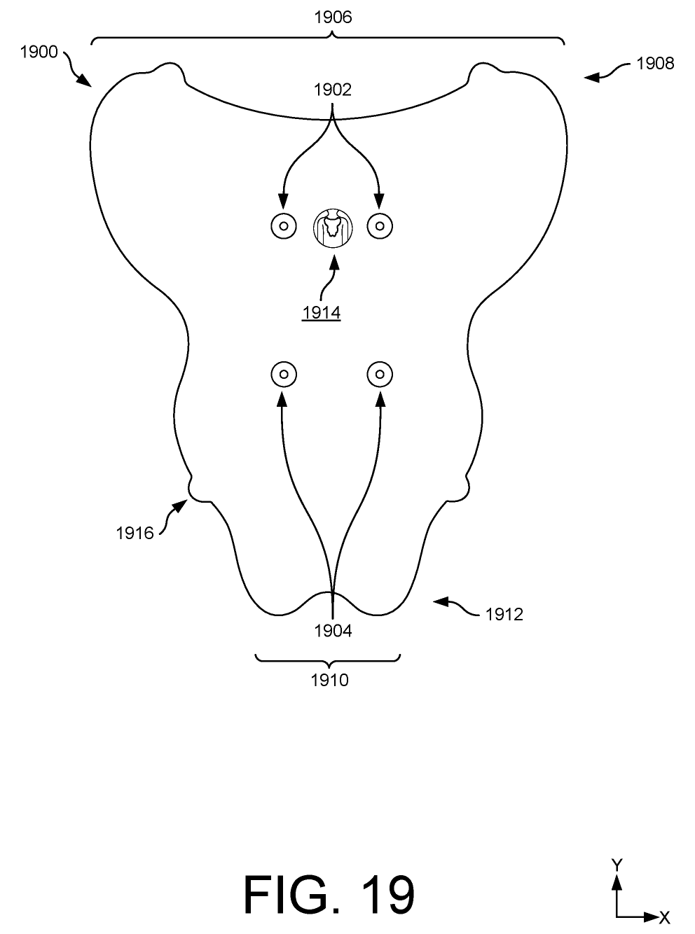

FIG. 19 illustrates an example pad 1900, according to an embodiment of the present disclosure. The pad 1900 may include first connectors 1902 and second connectors 1904. Each of the first connectors 1902 and the second connectors 1904 may be like the connectors 1102 for connecting to the connectors 520 of the stimulation device 102. Compared to pad 1100, the pad 1900 may receive two of the stimulation devices 102. For example, a first of the stimulation devices 102 may connect to the first connectors 1902, and a second of the stimulation devices 102 may connect to the second connectors 1904.

In some instances, the pad 1900 may be used for the upper back of the user. The pad 1900 may include a first dimension 1906 at a first end 1908 and a second dimension 1910 at a second end 1912. The first dimension 1906 may be greater than the second dimension 1910. The first end 1908 may be disposed adjacent to the upper back and/or neck of the user 1302, while the second end 1912 may be disposed adjacent to the middle of the back of the user 1302. The pad 1900 may also include various wings, arms, limbs, appendages, cutouts, notches, etc., to conform to the back of the user. An indicia 1914 may be disposed on the pad 1900 to indicate the type of the pad.

The stimulation devices 102 that connect to the first connectors 1902 and second connectors 1904 may be operated independently of one another and/or dependently of one another. Moreover, electrodes disposed on a bottom of the pad 1700 may be separated from one another. For example, the bottom of the pad 1700 may have four areas to accommodate the four electrodes of the first connectors 1902 and the second connectors 1904, respectively. Moreover, various tabs 1916 may extend from the pad 1700 to permit the pad 1700 to be removed from the user 1302. Moreover, in some instances, a first of the stimulation devices 102 may connect to the first connectors 1902 and the cable 1500 may be connected from the stimulation device 102 to the second connectors 1904.

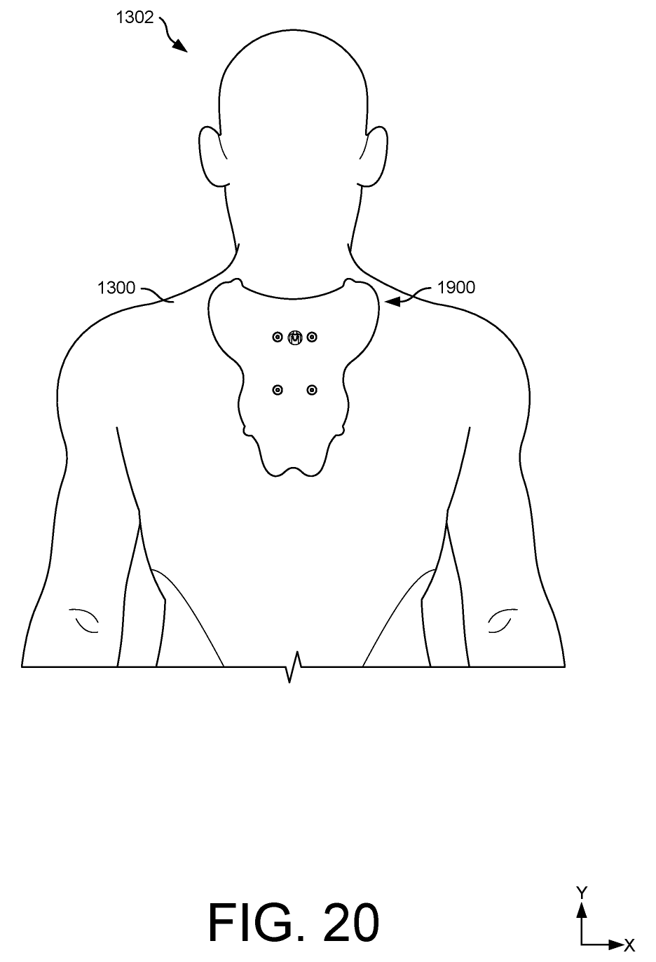

FIG. 20 illustrates the pad 1900 applied to the body 1300 of the user 1302, according to an embodiment of the present disclosure. As introduced above, the pad 1900 may be applied to the upper back of the user 1302.

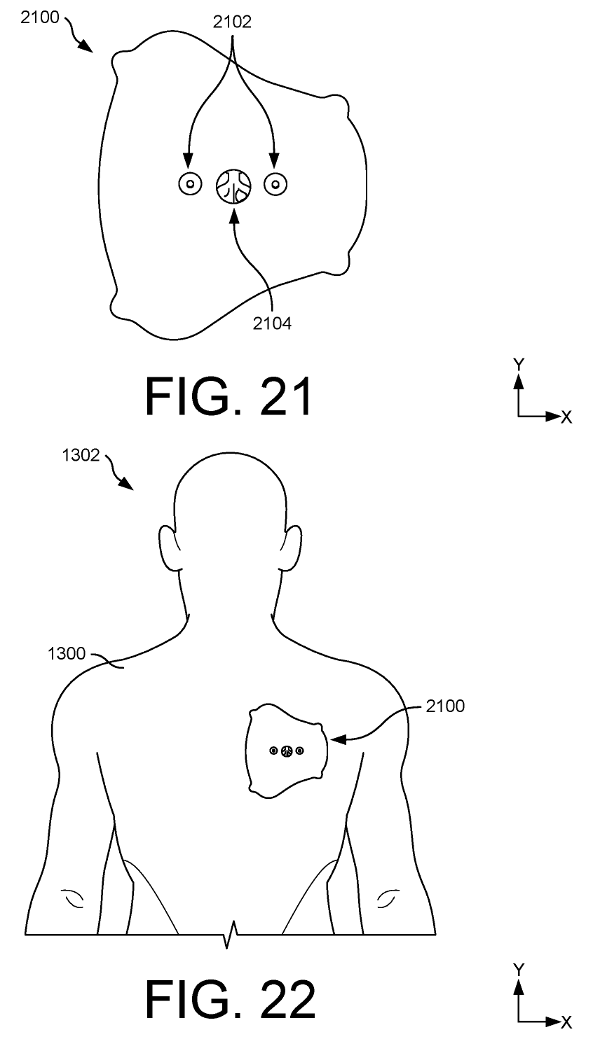

FIG. 21 illustrates an example pad 2100, according to an embodiment of the present disclosure. In some respects, the pad 2100 may be similar to the pad 1100. For example, the pad 2100 may include connectors 2102 for connecting to the connectors 520 of the stimulation device 102. However, as shown, the pad 2100 may include a different shape and/or may be a different type of pad than the pad 1100. In some instances, the pad 2100 may be used for a back and/or a shoulder of the user 1302 to provide TENS and/or EMS to the trapezius muscles and/or the rhomboid muscles of the user 1302. The shape of the pad 2100 accommodates a shape or contour of the shoulder and/or back of the user 1302. An indicia 2104 may be disposed on the pad 2100 to indicate the type of the pad.

FIG. 22 illustrates the pad 2100 applied to the body 1300 of the user 1302, according to an embodiment of the present disclosure. As introduced above, the pad 2100 may be applied to a back and/or a shoulder of the user 1302 to provide TENS and/or EMS to the trapezius muscles and/or the rhomboid muscles of the user 1302.

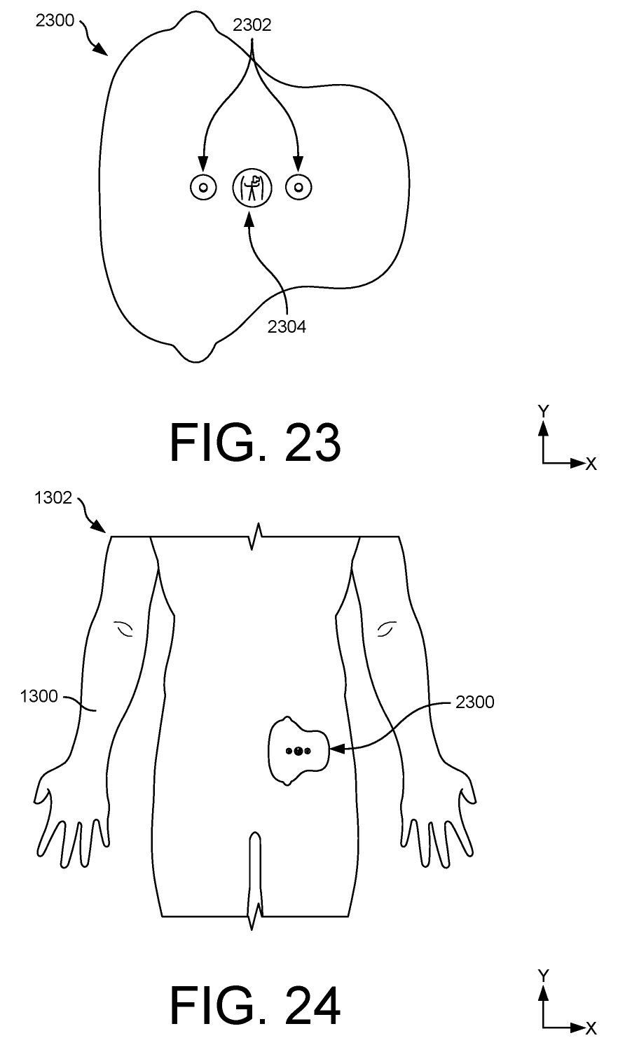

FIG. 23 illustrates an example pad 2300, according to an embodiment of the present disclosure. In some respects, the pad 2300 may be similar to the pad 1100. For example, the pad 2300 may include connectors 2302 for connecting to the connectors 520 of the stimulation device 102. However, as shown, the pad 2100 may include a different shape and/or may be a different type of pad than the pad 1100. In some instances, the pad 2300 may be used for a buttocks to provide TENS and/or EMS to the glutes of the user 1302. The shape of the pad 2300 accommodates a shape of the buttocks of the user 1302. An indicia 2304 may be disposed on the pad 2300 to indicate the type of the pad.

FIG. 24 illustrates the pad 2300 applied to the body 1300 of the user 1302, according to an embodiment of the present disclosure. As introduced above, the pad 2300 may be applied to the buttocks to provide TENS and/or EMS to the glutes of the user 1302.

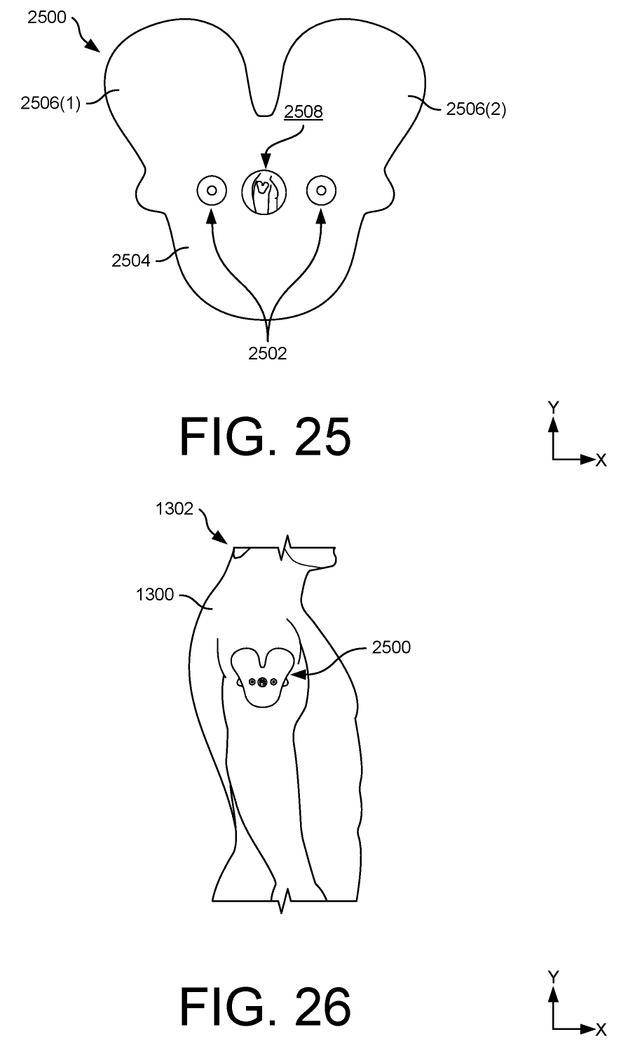

FIG. 25 illustrates an example pad 2500, according to an embodiment of the present disclosure. In some respects, the pad 2500 may be similar to the pad 1100. For example, the pad 2500 may include connectors 2502 for connecting to the connectors 520 of the stimulation device 102. However, as shown, the pad 2500 may include a different shape and/or may be a different type of pad than the pad 1100. In some instances, the pad 2500 may be used for the shoulder and/or upper arm of the user 1302. The pad 2500 may include a central portion 2504 and arms 2506 that extend from the central portion 2504, such as a first arm 2506(1) and a second arm 2506(2). The connectors 2502 may be located on the central portion 2504. The shape of the pad 2500 accommodates a shape or contour of the shoulder and/or upper arm of the user 1302. An indicia 2508 may be disposed on the pad 2500 to indicate the type of the pad.

FIG. 26 illustrates the pad 2500 applied to the body 1300 of the user 1302, according to an embodiment of the present disclosure. As introduced above, the pad 2500 may be applied to the shoulder and/or upper arm of the user 1302.

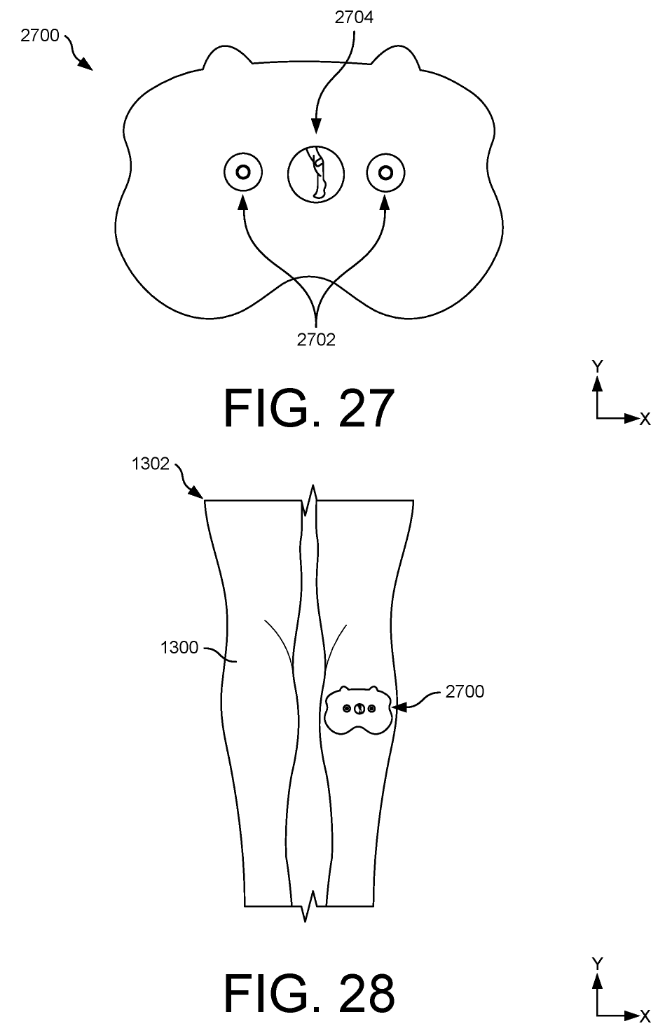

FIG. 27 illustrates an example pad 2700, according to an embodiment of the present disclosure. In some respects, the pad 2700 may be similar to the pad 1100. For example, pad 2700 may include connectors 2702 to connect to the connectors 520 of stimulation device 102. However, as shown, the pad 2700 may include a different shape and/or a different type of pad than the pad 1100. In some instances, the pad 2700 may be used to provide TENS and/or EMS to the calf of the user 1302. The shape of the pad 2700 accommodates the shape of the calf of the user 1302. An indicia 2704 may be disposed on the pad 2700 to indicate the type of the pad.

FIG. 28 illustrates the pad 2700 applied to the body 1300 of the user 1302, according to an embodiment of the present disclosure. As introduced above, the pad 2700 may be applied to the calf of the user 1302.

Although FIGS. 16-27 illustrate example pads, with different shapes and contours, other pads are envisioned. For example, pads with other shapes and contours may be used for the hip, thigh, neck, abdomen, etc. Regardless of the specific shapes and contours, the pads include connections for receiving and connecting to the stimulation devices. The areas of the pads that include the electrodes may also be sized based at least in part on the size of the pad, and/or the number of electrodes.

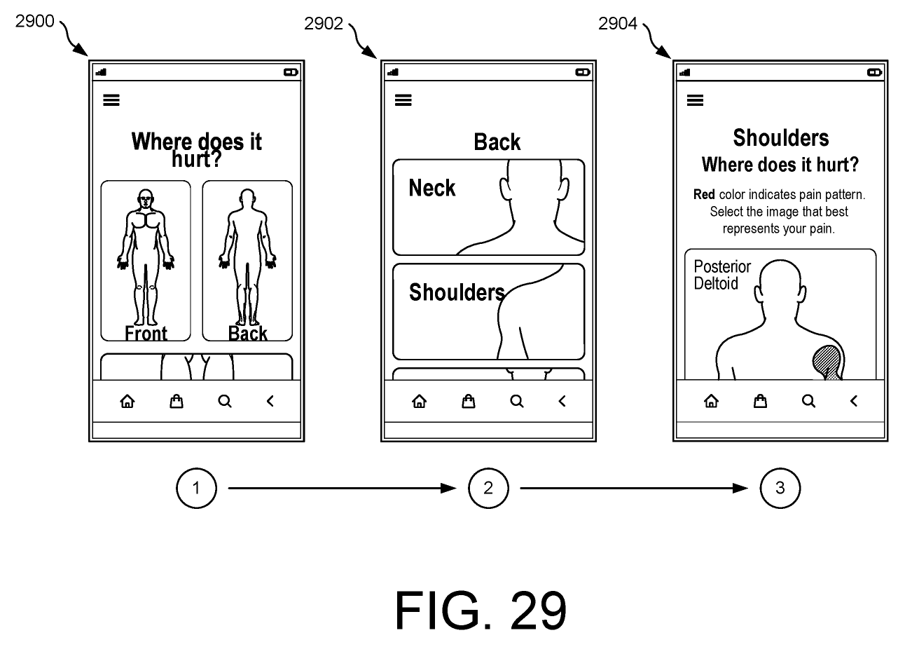

FIGS. 29-31 illustrate a sequence of UIs for guiding the user through the placement and operation of the stimulation device 102, according to an embodiment of the present disclosure. Introduced above, the user 1302 may place the device 110 in proximity to the case 106, such as the RFID 242 in the case 106, as denoted by the icon 732, to surface UIs on the device 110 for guiding the user 1302 to set up the pads and the stimulation devices 102.

For example, in response to placing the device 110 in proximity to the RFID 242, the device 110 at “1” in FIG. 29 may display a first UI 2900. The first UI 2900 may display portions of a body, asking the user 1302 where the pain, discomfort, etc., is present. The user 1302 may scroll on the first UI 2900 to additional portions of the user 1302. For example, the first UI 2900 may display a front, a back, feet, etc., of the body. The user 1302 may provide an input (e.g., touch) to a portion of the first UI 2900 corresponding to where the pain, discomfort, etc., is present.

A second UI 2902 may be displayed at “2” in FIG. 29. For example, in response to receiving input at the first UI 2900, the second UI 2902 may be displayed. The second UI 2902 may display portions of the body 1300, asking the user 1302 where the pain, discomfort, etc., is present. However, the portions displayed in the second UI 2902 may be more specific than those in the first UI 2900. For example, if the user 1302 has selected the “back” from the first UI 2900, the second UI 2902 may display a neck, a shoulder, a back, legs, buttocks, calves, etc., of the body. The user 1302 may scroll through the different options to select where the pain, discomfort, etc., is present

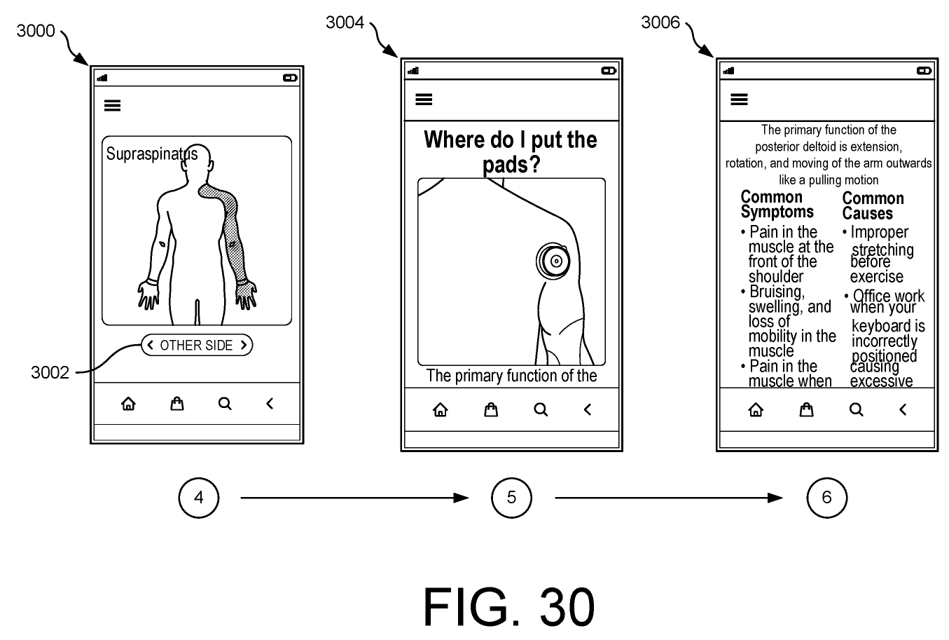

A third UI 2904 may be displayed at “3” in FIG. 29. For example, in response to receiving input at the second UI 2902, the third UI 2904 may be displayed. The third UI 2904 may more specifically ask the user where the pain, discomfort, etc., is present. For example, after selecting “shoulders” from the second UI 2902, the third UI 2904 may more pointedly ask the user 1302 where the pain, discomfort, etc., is present. In some instances, the third UI 2904 may illustrate the associated pain (e.g., Posterior Deltoid). From the third UI 2904, the user 1302 may scroll to display a fourth UI 3000, which is shown at “4” in FIG. 30. The fourth UI 3000 may still be associated with the shoulder, but the fourth UI 3000 may illustrate the associated pain (e.g., Supraspinatus). In other words, from the third UI 2904 and the fourth UI 3000, the user 1302 may indicate whether the source of their pain is associated with the Posterior Deltoid or the Supraspinatus.

Moreover, the fourth UI 3000 (or the third UI 2904) may allow the user to switch sides of their body where the pain, discomfort, etc., is present. For example, the third UI 2904 and the fourth UI 3000 show the pain originating from the right sight of the body. However, an icon 3002 may be displayed that allows the user 1302 to switch sides. Providing input to the icon 3002 may switch the illustrations of the user from the right side to the left side.

After making a selection from the third UI 2904 or the fourth UI 3000, a fifth UI 3004 may be displayed at “5” in FIG. 30. For example, the user 1302 may make a selection of the “Posterior Deltoid” from the third UI 2904. In response, the fifth UI 3004 may be displayed, where the fifth UI 3004 may instruct or otherwise guide the user 1302 regarding the placement of the pad 1100 and/or the stimulation device 102 on their body. In some instances, the fifth UI 3004 may indicate the type of pad (e.g., the pad 1100, the pad 1700, the pad 2700, etc.) to use to treat the pain, discomfort, etc. The type of pad may be based on the previous inputs to the UIs (e.g., from the first UI 2900, the second UI 2902, the third UI 2904, etc.). In some instances, the fifth UI 3004 may provide a video, GIF, etc., for guiding the user 1302 in placing the pad 1100.

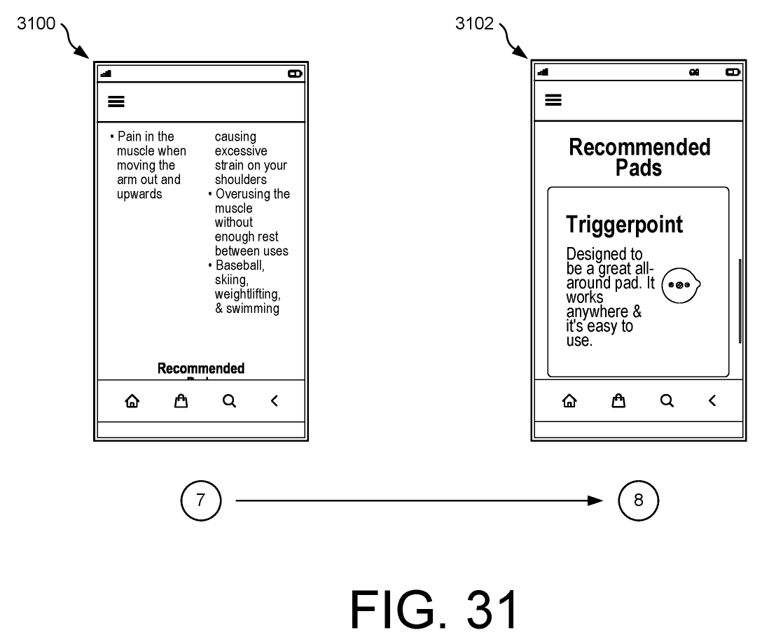

The user 1302 may scroll on the fifth UI 3004 to display a sixth UI 3006 at “6” in FIG. 30. The sixth UI 3006 illustrates symptoms and/or causes of the pain, discomfort, etc. For example, the sixth UI 3006 may list causes of the pain, discomfort, etc., felt in the Posterior Deltoid of the user 1302. The user may continue scrolling to a seventh UI 3100 at “7” in FIG. 31. The seventh UI 3100 may list additional symptoms and causes. The user 1302 may continue to scroll to an eighth UI 3102 at “8” in FIG. 31. The eighth UI 3102 may list recommended pads for the user 1302 to treat their pain, discomfort, etc., in the Posterior Deltoid.

Although FIGS. 29-31 illustrate certain UIs, additional or alternative UIs are envisioned. For example, a UI may be presented that indicates the mode(s) 224 and/or setting(s) 226 by which to control the stimulation device 102. Moreover, UIs may be presented that allow the user 1302 to indicate the severity of their pain (e.g., on a scale from 1 to 10), which may impact the placement or recommendation of the pads to use, the mode(s) 224, and/or the setting(s) 226. Moreover, although the UIs illustrate the placement of one of the pads, the UIs may guide the user 1302 to place more than one of the pads and/or how to independently or dependently control the pads.

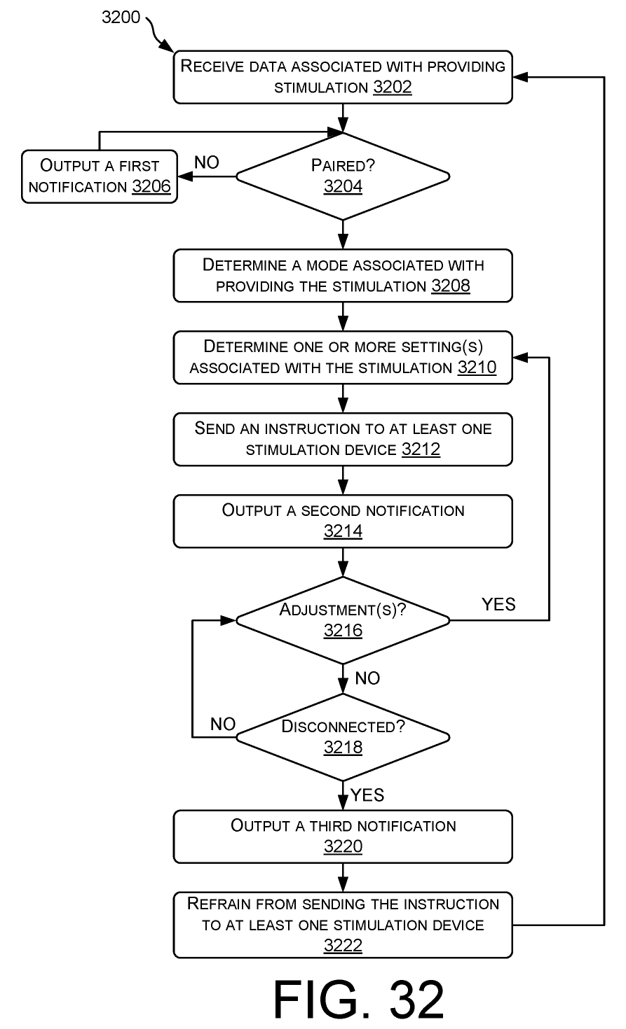

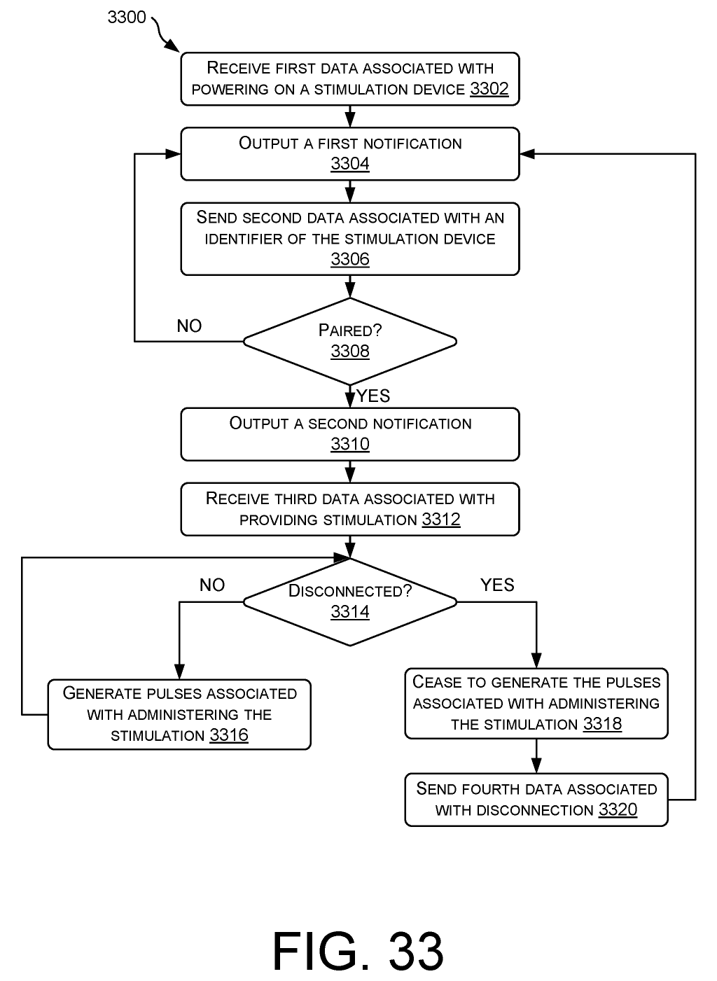

FIGS. 32 and 33 illustrate example processes associated with providing TENS and/or EMS, according to examples of the present disclosure. The processes described herein are illustrated as collections of blocks in logical flow diagrams, which represent a sequence of operations, some or all of which may be implemented in hardware, software, or a combination thereof. In the context of software, the blocks may represent computer-executable instructions stored on one or more computer-readable media that, when executed by one or more processors, program the processors to perform the recited operations. Generally, computer-executable instructions include routines, programs, objects, components, data structures and the like that perform particular functions or implement particular data types. The order in which the blocks are described should not be construed as a limitation, unless specifically noted. Any number of the described blocks may be combined in any order and/or in parallel to implement the processes, or alternative processes, and not all of the blocks need to be executed. For discussion purposes, the processes are described with reference to the environments, devices, architectures, diagrams, and systems described in the examples herein, such as those described with respect to FIGS. 1-31, although the processes may be implemented in a wide variety of other environments, architectures, and systems.

FIG. 32 illustrates an example process 3200 associated with providing TENS and/or EMS to the user, according to an embodiment of the present disclosure.

At 3202, the process 3200 may include receiving data associated with providing stimulation. In some instances, the data may be received with the first button 310 of the remote control 104. For example, the user may press and hold the first button 310 to power on the remote control 104. The stimulation provided may be TENS or EMS.

At 3204, the process 3200 may include determining whether the remote control is paired with stimulation devices. For example, whether the remote control 104 is paired with the stimulation devices 102 may be based on the stimulation devices 102 being powered on and/or in proximity to the remote control 104. Moreover, the remote control 104 may be paired with more than one of the stimulation devices 102. If At 3204 the process 3200 determines that the remote control 104 is not paired with the stimulation devices 102, the process 3200 may follow the “NO” route and proceed to 3206.

At 3206 the process 3200 may include outputting a first notification. For example, the display 228 of the remote control 104 may output a first notification associated with the remote control 104 not being paired, or recognizing, the stimulation devices 102. In some instances, the first notification may also provide instructions to pair the remote control 104 with the stimulation device 102, such as to power on the stimulation device 102, place the stimulation device 102 in proximity to the remote control 104, and so forth. From 3206, the process 3200 may proceed to 3204 to determine whether the remote control 104 is paired with the stimulation device 102. At 3204, if the process 3200 determines that the remote control 104 is paired with the stimulation device 102, the process 3200 may follow the “YES” route and proceed to 3208.

At 3208, the process 3200 may include determining a mode associated with providing the stimulation. The user 1302, for example, may use the second button 312 to scroll through the different modes to select a mode of the mode(s) 224. As introduced above, the mode(s) 224 may be associated with different patterns, cycles, etc., of the electrical pulses. The user 1302 may utilize the first button 310 to select the mode(s) 224 that is desired.

At 3210, the process 3200 may include determining one or more setting(s) associated with stimulation. The setting(s) 226 may include a duration, timing, intensity, frequency, etc. of the stimulation. In some instances, the setting(s) 226 may be based on the mode 224 selected by the user 1302.

At 3212, the process 3200 may include sending an instruction to at least one stimulation device. For example, the remote control 104 may send the instruction(s) 208 to at least one of the stimulation devices 102. The instruction(s) 208 may indicate the mode 224 and/or the setting(s) 226 associated with generating the electrical pulses.

At 3214, the process 3200 may include outputting a second notification. For example, the remote control 104 may output a second notification on the display 228. In some instances, the notification may include an indication of the mode 224, the setting(s) 226, an amount of time remaining, etc.

At 3216, the process 3200 may include determining whether any adjustment(s) are received. For example, using the remote control 104, such as the second button 312, the user 1302 may increase the intensity of the electrical pulses, the duration, the frequency, etc. In this sense, the user 1302 may modify the mode(s) 224 and/or the setting(s) 226 according to their desired preferences. If at 3216 adjustment(s) are received, the process 3200 may follow the “YES” route and proceed to 3210. Alternatively, if adjustment(s) are not received, the process 3200 may follow the “NO” route and proceed to 3218.

At 3218, the process 3200 may include determining whether the at least one stimulation device is disconnected. For example, the pad adhered to the user 1302 may be removed and/or the stimulation device 102 may lose connection with the pad. Here, the connectors 520 of the stimulation device 102 may be decoupled or disconnected from the connectors 1102 of the pad 1100, for example. In such instances, the stimulation device 102 may refrain or cease to output the electrical pulses. If the connection is interrupted, the remote control 104 may receive an indication of such (e.g., from the stimulation device 102). If the connection is present, the process 3200 may follow the “NO” route and loop to 3216. Alternatively, if the connection is disconnected, the process 3200 may follow the “YES” route and proceed to 3220.

At 3220, the process 3200 may include outputting a third notification. For example, the remote control 104 may output a notification that the pad was removed, or that the connection was otherwise interrupted. In some instances, the notification may also indicate how to reconnect the pad and/or how to reconnect the stimulation device to the pad.

At 3222, the process 3200 may include refraining from sending the instruction to the at least one stimulation device. For example, when disconnected, the remote control 104 may refrain from sending the instruction(s) 208 or may send an instruction(s) 208 to the stimulation device 102, instructing the stimulation device 102 to refrain from outputting the electrical pulses. From 3222, the process 3200 may proceed to 3202.

FIG. 33 illustrates an example process 3300 associated with providing TENS and/or EMS to the user, according to an embodiment of the present disclosure.

At 3302, the process 3300 may include receiving data associated with powering on a stimulation device. In some instances, the data may be received with the button 506 of the stimulation device 102. For example, the user may press and hold the button 506 to power on the stimulation device 102.

At 3304, the process 3300 may include outputting a first notification. For example, the light ring 508 (or the I/O component(s) 210 of the stimulation device 102) may output a first notification. In some instances, the first notification may include a first pattern, color, sequence, etc. In some instances, the first notification may be associated with the stimulation device 102 being ready to be paired with the remote control 104.

At 3306, the process 3300 may include sending second data associated with an identifier of the stimulation device. For example, the stimulation device 102 may send data associated with an identifier to the remote control 104. The data may be used to pair the remote control 104 and the stimulation device 102.

At 3308, the process 3300 may include determining whether the stimulation device is paired. For example, the stimulation device 102 may determine whether its paired with the remote control 104. In some instances, the stimulation device 102 may receive an acknowledgement (or data) from the remote control 104 confirming that its paired with the remote control 104. If the process 3300 determines that the stimulation device 102 is not paired with the remote control 104, the process 3300 may follow the “NO” route and proceed to 3304. Alternatively, if the process 3300 determines that the stimulation device 102 is paired with the remote control 104, the process 3300 may follow the “YES” route and proceed to 3310.

At 3310, the process 3300 may include outputting a second notification. For example, the light ring 508 (or the I/O component(s) 210 of the stimulation device 102) may output a second notification. In some instances, the second notification may include a second pattern, color, sequence, etc., different from the first notification. In some instances, the first notification may be associated with the stimulation device 102 being paired with the remote control 104.

At 3312, the process 3300 may include receiving third data associated with providing stimulation. For example, stimulation device 102 may receive instruction(s) 208 from the remote control 104, where the instruction(s) 208 indicate the mode(s) 224, the setting(s) 226, etc., of the stimulation to be provided.

At 3314, the process 3300 may include determining whether the stimulation device and/or the pad is disconnected. Whether the stimulation device 102 is disconnected may be based on the stimulation device 102 being disconnected from the pad 1100. For example, the connectors 520 of the stimulation device 102 may be decoupled or disconnected from the connectors 1102 of the pad 1100. Alternatively, the pad may be removed from the user. If the pad is removed from the user, the circuit between the electrodes of the pad is no longer present. In such instances, the stimulation device 102 may receive an indication associated with the interruption of the circuit. If at 3314 the process 3300 determines that the stimulation device and/or the pad is not disconnected, the process 3300 may follow the “NO” route and proceed to 3316.

At 3316, the process 3300 may include generating pulses associated with administering the stimulation. For example, the pulse generator 204 may generate the electrical pulses delivered to the user. The pulse generator 204 may generate the electrical pulses in associated with the instruction(s) 208 received from the remote control 104. In some instances, the stimulation device 102 may receive a single instruction at the outset of providing the electrical pulses, where the instruction indicates the intensity, frequency, duration, etc., of the electrical pulses. Alternatively, the stimulation device 102 may continuously receive the instruction(s) 208 for administering the electrical pulses. Moreover, the stimulation device 102 may receive the instruction(s) 208 based upon adjustment(s) being received at the remote control 104 (e.g., increase frequency, duration, mode, etc.). From 3316, the process 3300 may loop to 3314.

Returning to 3314, if the process 3300 determines that the stimulation device and/or the pad is disconnected, the process 3300 may follow the “YES” route and proceed to 3318.

At 3318, the process 3300 may include ceasing to generate the pulses associated with administering the stimulation. For example, if the stimulation device 102 and/or the pad is disconnected, conductivity may not exist between the electrodes of the pad and/or between the connectors 520 of the stimulation device 102 and the pad. In such instances, the pulse generator 204 may refrain or cease from generating the electrical pulses.

At 3320, the process 3300 may include sending fourth data associated with the disconnection. For example, when the stimulation device and/or the pad becomes disconnected, the stimulation device 102 may send an indication of such to the remote control 104. In response, the remote control 104 may output an indication on the display 228, for example, that the stimulation is paused. From 3320, the process 3300 may loop to 3304.

While various examples and embodiments are described individually herein, the examples and embodiments may be combined, rearranged, and modified to arrive at other variations within the scope of this disclosure.

Although the subject matter has been described in language specific to structural features and/or methodological acts, it is to be understood that the subject matter defined in the appended claims is not necessarily limited to the specific features or acts described. Rather, the specific features and acts are disclosed as illustrative forms of implementing the claims

What is claimed is:

1. A system comprising:
a case including:
a first receptacle,
a second receptacle,
a third receptacle,
first connector pins disposed within the first receptacle,
second connector pins disposed within the second receptacle,
third connector pins disposed within the third receptacle,
first connectors disposed within the first receptacle,
second connectors disposed within the second receptacle, and
a radio-frequency identification;
a first stimulation device to be disposed within the first receptacle, the first stimulation device including:
a button,
a light ring disposed around the button of the first stimulation device,
connectors, wherein individual connectors of the first stimulation device engage with individual connectors of the first connectors,
connector pads, wherein a portion of the connector pads of the first stimulation device engage with individual connector pins of the first connector pins, and
a pulse generator;
a second stimulation device configured to be disposed within the second receptacle, the second stimulation device including:
a button,
a light ring disposed around the button of the second stimulation device,
connectors, wherein individual connectors of the second stimulation device engage with individual connectors of the second connectors,
connector pads, wherein a portion of the connector pads of the second stimulation device engage with individual connector pins of the second connector pins, and
a pulse generator; and
a remote control disposed within the third receptacle, the remote control including connector pads, wherein a portion of the connector pads of the remote control engage with individual connector pins of the third connector pins.

2. The system of claim 1, wherein:
the first stimulation device includes four of the connector pads;
two of the connector pads of the first stimulation device engage with the individual connector pins of the first connector pins;
the second stimulation device includes four of the connector pads;
two of the connector pads of the second stimulation device engage with the individual connector pins of the second connector pins;
the remote control includes four of the connector pads; and
two of the connector pads of the remote control engage with the individual connector pins of the third connector pins.

3. The system of claim 1, further comprising a pad including connectors, wherein at least one of:
individual connectors of the first stimulation device engage with individual connectors of the pad; or
individual connectors of the second stimulation device engage with individual connectors of the pad.

4. The system of claim 3, wherein:
a connection formed between the individual connectors of the first stimulation device and the individual connectors of the pad transfers electrical current; or
a connection formed between the individual connectors of the second stimulation device and the individual connectors of the pad transfers electrical current.

5. The system of claim 1, wherein:
the first connectors include at least one of a magnetic element or a ferrous material;
the second connectors include at least one of a magnetic element or a ferrous material;
the connectors of the first stimulation device include at least one of a magnetic element or a ferrous material; and
the connectors of the second stimulation device include at least one of a magnetic element or a ferrous material.

6. The system of claim 1, wherein:
the first stimulation device includes a wireless interface;
the second stimulation device includes a wireless interface; and
the remote control includes a wireless interface for communicating with the wireless interface of the first stimulation device and the wireless interface of the second stimulation device.

7. The system of claim 1, wherein:
the remote control includes:
one or more processors; and
one or more non-transitory computer-readable media storing computer-executable instructions that, when executed by the one or more processors of the remote control , cause the one or more processors of the remote control to perform operations comprising:
receiving first data associated with providing stimulation,
determining, based at least in part on the first data, a setting associated with the stimulation, and
sending second data associated with the setting to the first stimulation device; and
the first stimulation device includes:
one or more processors, and
one or more non-transitory computer-readable media storing computer-executable instructions that, when executed by the one or more processors of the first stimulation device, cause the one or more processors of the first stimulation device to perform operations comprising:
receiving the second data, and
causing, based at least in part on the second data, the pulse generator of the first stimulation device to generate electrical pulses.

8. A system comprising:
a remote control including:
a display,
a button,
one or more processors, and
one or more non-transitory computer-readable media storing computer-executable instructions that, when executed by the one or more processors of the remote control, cause the one or more processors of the remote control to perform operations comprising:
receiving, via the button, first data associated with providing electrical stimulation,
determining at least one of a mode or a setting associated with providing the electrical stimulation,
displaying, on the display, an indication of the at the least one of the mode or the setting, and
sending second data associated with the at the least one of the mode or the setting; and
a stimulation device including:
a pulse generator,
one or more processors, and
one or more non-transitory computer-readable media storing computer-executable instructions that, when executed by the one or more processors of the stimulation device, cause the one or more processors of the stimulation device to perform operations comprising:
receiving the second data from the remote control, and
generating, based at least in part on the second data and via the pulse generator, electrical pulses to provide the electrical stimulation.

9. The system of claim 8, wherein the at least one of the mode or the setting includes at least one of a frequency, a duration, or an intensity of the electrical stimulation.

10. The system of claim 8, wherein:
the operations of the remote control further comprise:
receiving third data associated with providing second electrical stimulation,
determining at least one of a second mode or a second setting associated with providing the second electrical stimulation, and
sending fourth data associated with the at the least one of the second mode or the second setting; and
the operations of the stimulation device further comprise:
receiving the fourth data from the remote control, and
generating, based at least in part on the fourth data and via the pulse generator, second electrical pulses to provide the second electrical stimulation.

11. The system of claim 8, further comprising a second stimulation device including:
a pulse generator;
one or more processors; and
one or more non-transitory computer-readable media storing computer-executable instructions that, when executed by the one or more processors of the second stimulation device, cause the one or more processors of the second stimulation device to perform operations comprising:
receiving third data from the remote control, and
generating, based at least in part on the third data and via the pulse generator, second electrical pulses to provide the electrical stimulation.

12. The system of claim 11, wherein the second electrical pulses are different than the electrical pulses.

13. The system of claim 8, wherein:
the stimulation device includes connectors;
individual connectors of the connectors of the stimulation device engage with individual connectors of connectors of a pad, the pad being adhered to a user; and
the operations of the stimulation device further comprise:
determining that at least one of:
the individual connectors of the stimulation device are engaged with the individual connectors of the pad, or
conductivity exists between the connectors of the stimulation device; and
based at least in part on determining that the at least one of the individual connectors of the stimulation device are engaged with the individual connectors of the pad or that the conductivity exists between the connectors of the stimulation device, causing the pulse generator to generate the electrical pulses.

14. The system of claim 13, the operations of the stimulation device further comprising:
determining that the at least one of:
the individual connectors of the stimulation device are disengaged with the individual connectors of the pad, or
conductivity is lost between the connectors of the stimulation device; and
based at least in part on determining that the at least one of the individual connectors of the stimulation device are disengaged with the individual connectors of the pad or that the conductivity is lost between the connectors of the stimulation device, causing the pulse generator to cease generating the electrical pulses.

15. A system comprising:
a remote control; and
a stimulation device including:
a pulse generator,
one or more processors, and
one or more non-transitory computer-readable media storing computer-executable instructions that, when executed by the one or more processors, cause the stimulation device to perform operations comprising:
receiving data from the remote control associated with providing electrical stimulation;
determining, based at least in part on the data, a setting associated with providing the electrical stimulation, and
generating, based at least in part on the setting and via the pulse generator, electrical pulses to provide the electrical stimulation.

16. The system of claim 15, wherein:
the stimulation device is configured to connect to a pad worn by a user; and
the operations further comprise:
determining that the stimulation device is connected to the pad or that conductivity exists between the pad and the user, and
based at least in part on the stimulation device being connected to the pad or conductivity exists between the pad and the user, causing the pulse generator to generate the electrical pulses.

17. The system of claim 16, wherein the operations further comprise:
determining that the stimulation device is disconnected from the pad or that a lack of conductivity between the pad and the user; and
based at least in part on the stimulation device being disconnected from the pad or the lack of conductivity between the pad and the user, causing the pulse generator to refrain from generating the electrical pulses.

18. The system of claim 15, wherein the operations further comprise:
receiving second data from a device associated with providing second electrical stimulation;
determining, based at least in part on the second data, a second setting associated with providing the second electrical stimulation; and
generating, based at least in part on the second setting and via the pulse generator, second electrical pulses to provide the second electrical stimulation.

19. The system of claim 15, wherein:
the stimulation device includes a light ring; and
the operations further comprise:
causing, at a first instance in time output of a first notification via the light ring, and
causing, at a second instance in time and based at least in part on generating the electrical pulses, output of a second notification via the light ring, the second notification being different than the first notification.

20. The system of claim 15, further comprising a case to receive the remote control and the stimulation device, the case including a radio-frequency identification.
